(19) United States
(12) Patent Application Publication
(10) Pub. No.: US 2015/0164910 A1
(43) Pub. Date: Jun. 18, 2015

(54) ANTIVIRAL COMPOUNDS AND METHODS OF USE

(71) Applicant: The Regents of the University of Califonia, Oakland, CA (US)

(72) Inventors: Paul Krogstad, Culver City, CA (US); Jun Zuo, Los Angeles, CA (US); Steve H. Kye, Glendora, CA (US); Robert D. Damoiseaux, Beverly Hills, CA (US)

(21) Appl. No.: 14/407,665

(22) PCT Filed: Jun. 14, 2013
(86) PCT No.: PCT/US13/46024

§ 371 Date: Dec. 12, 2014

Related U.S. Application Data
(60) Provisional application No. 61/660,407, filed on Jun. 15, 2012.

(51) Int. Cl. A61K 31/55; A61K 31/496; A61K 31/4709; A61K 31/453; A61K 31/47; A61K 31/538; A61K 31/4184; A61K 31/433; A61K 31/437; A61K 31/5377

(52) U.S. Cl. CPC A61K 31/55; A61K 31/437; A61K 31/496; A61K 31/4709; A61K 31/5377; A61K 31/47; A61K 31/538; A61K 31/4184; A61K 31/433; A61K 31/453

(57) ABSTRACT

Methods for treating enteroviral infections in cells are provided. Aspects of the methods include inhibiting the replication of enterovirus or replication of other virus and/or inhibiting cell death in a cell infected with enterovirus or other viruses.

### CROSS REFERENCE TO RELATED APPLICATIONS

This application claims priority under 35 U.S.C. §119(e) to U.S. Provisional Application No. 61/660,407, filed Jun. 15, 2012, which is incorporated herein in its entirety by reference.

### STATE OF THE ART

Picornaviruses are a family of small non-enveloped viruses with a single-stranded RNA genome surrounded by an icosahedral protein capsid. Most human picornavirus infections are due to members of the enterovirus genus, which includes polioviruses, rhinoviruses, coxsackieviruses, echoviruses, and sequentially numbered enteroviruses, beginning with enterovirus 68. Hepatitis A virus is also a member of the picornavirus family.

Enteroviruses are common human viruses associated with various clinical syndromes, from minor febrile illness to severe, potentially fatal conditions (e.g., aseptic meningitis, paralysis, myocarditis, and neonatal enteroviral sepsis). Multiple enterovirus serotypes exist. Individual serotypes have different temporal patterns of circulation and often are associated with different clinical manifestations. Changes in circulating serotypes might be accompanied by large-scale outbreaks. There is a need in the art for more effective therapies to treat individuals infected with enteroviruses.

### SUMMARY

This invention relates generally to methods for treating viral infections in general and enterovirus infections in specific. Provided are small molecules of therapeutic value which inhibit the replication of enteroviruses and may have applications for other viruses as well. Furthermore, these compounds are useful as well for research using cell culture systems wherein the cell is infected with enterovirus or other viruses. In addition we claim methods comprising contacting the cells/virus in vivo or in vitro with an effective amount of a compound selected from the compounds described herein. In some embodiment, it is contemplated that a compound of Formula I as disclosed herein below is administered, in combination with a compound of Formula IV as disclosed herein below, for treating viral infections in general and enterovirus infections in specific. Specific compounds of Formula I useful as above include compounds 1-21 as described in Table 1. Specific compounds of Formula IV useful as above include compounds 44-46 as described in Table 1. Unless specified otherwise, a compound as used herein, includes its tautomers, and pharmaceutically acceptable salts of each thereof. The compounds can be delivered in a pharmaceutically acceptable excipient to a subject or patient in need thereof.

### BRIEF DESCRIPTION OF THE DRAWINGS

FIG. 1 depicts the RT-PCR data described in Example 3. RT-PCR reveals marked reduction in viral RNA in cells treated with selected hit compounds.

FIG. 2 depicts RT-PCR data and immunoblot data for 10 μM of Fluoxetine, as described in Example 3. FIG. 2A depicts RT-PCR detection of CVB3 RNA in cells treated with guanidine (Guan) of fluoxetine (Fluox) beginning 30 min before inoculation of cell cultures. FIG. 2B depicts Immunoblot detection of viral capsid protein six hours after infection with CVB3-H3; no viral protein is detected in cells treated with fluoxetine (Fluox) and norfluoxetine (Norf). Immunoblot detection of glycyl tRNA synthease protein (GlyRS) (a house keeping protein) is shown as a loading control.

FIG. 3 shows photomicrographs of cells infected with eGFP-expressing recombinant CVB3 and treated with the indicated compounds as described in Example 4.

FIG. 4 shows quantitation of antiviral activity of fluoxetine (Fluox) against additional enteroviruses by plaque reduction described in Example 5.

FIG. 5 shows immunoblot detection of CVB3 protein as described in Example 6.

FIG. 6 shows antiviral activities of compound 3 against poliovirus as described in Example 7.

FIG. 7 shows that time of addition experiment demonstrates compound 45 should be present at the time of infection to reduce the amount of virus produced six hours after infection, while compound 3 and mefloquine are equivalently active even if added to culture 2 hours after infection begins, as described in Example 8.

### DETAILED DESCRIPTION

### Definitions

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)).

As used herein, certain terms may have the following defined meanings. As used in the specification and claims, the singular form “a,” “an” and “the” include singular and plural references unless the context clearly dictates otherwise.

The term “comprising” is intended to mean that the compounds and methods include the recited elements, but not excluding others. “Consisting essentially of” when used to define compositions and methods, shall mean excluding other elements of any essential significance to the compounds or methods. “Consisting of” shall mean excluding more than trace elements of other ingredients for claimed compounds and substantial method steps. Embodiments defined by each of these transitional terms are within the scope of this invention. Accordingly, it is intended that the processes and compositions can include additional steps and components (comprising) or alternatively include additional steps and compounds of no significance (consisting essentially of) or alternatively, intending only the stated methods steps or compounds (consisting of).

Mammals, subjects or patients include, but are not limited to, murines, rats, rabbits, simians, bovines, ovines, porcines, canines, felines, farm animals, sport animals, pets, equines, and primates, particularly humans.

As used herein, “treating” or “treatment” of a disease, disorder, symptom or condition will depend on the disease, disorder, symptom or condition to be treated, and the mammal to be treated. In general, treatment intends one or more of inhibiting the progression of the manifested disease, disorder, symptom or condition as measured by clinical or sub-clinical parameters (where the term “inhibiting” or “inhibition” is intended to be a subset of “treating” or “treatment”), arresting the development of the disease, disorder, symptom or condition as measured by clinical or sub-clinical parameters, ameliorating or causing regression of the disease, disorder, symptom or condition as measured by clinical or sub-clinical parameters, or reducing pain or discomfort for the mammal treated as measured by clinical and/or pharmacological parameters.

As used herein, “inhibit,” “inhibiting,” “reduce” or “reducing” or any variation of these terms includes any measurable decrease or complete inhibition to achieve a desired result such as but not limited to inhibition of viral entry, replication, budding, transcription, splicing, genome editing etc.

As used herein, a “therapeutically effective amount” or an “effective amount” is used synonymously with and intends an amount sufficient to effect beneficial or desired results. An effective amount can be administered in one or more administrations, applications, or dosages.

As used herein, the term in vitro administration refers to manipulations performed on cells removed from or outside of a subject, including, but not limited to cells in culture.

The term in vivo administration includes all manipulations performed within a subject, including administrations.

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs).

As used herein, the term “derivative” of a compound refers to a chemically modified compound wherein the chemical modification takes place either at a functional group of the compound, aromatic ring, or carbon backbone; including, for example, esters of alcohol-containing compounds, esters of carboxyl-containing compounds, amides of amine-containing compounds, amides of carboxyl-containing compounds, imines of amino-containing compounds, and the like.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases well known to those skilled in the art.

As used herein, the term “administration” refers to the act of giving a drug, prodrug, or other agent, or therapeutic treatment to a physiological system (e.g., a subject or in vivo, in vitro, or ex vivo cells, tissues, and organs). Illustrative routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intraperitoneally, into cerebrospinal fluid, etc.) and the like.

Administration in “combination” refers to the use of two or more drugs in therapy, i.e., use of two or more compounds as utilized herein, and optionally other antiviral agent(s), to treat viral infections in general and enterovirus infections in specific. Administration in “combination” refers to the administration of two or more agents in any manner in which the pharmacological effects of each are manifest in the patient at the same time. Thus, administration in combination does not necessarily require that a single pharmaceutical composition, the same dosage form, or the same route of administration be used for administration of both agents or that the two agents be administered at precisely the same time.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy haloalkoxy, haloalkenyloxy, haloalkynyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acyl, alkenylacyl, alkynylacyl, arylacyl, acylamino, acyloxy, aldehydo, alkylsulphonyl, arylsulphonyl, alkylsulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, alkylthio, arylthio, acylthio and the like.

The salts of the compounds described herein are in certain embodiments, pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicyclic, sulphanilic, aspartic, glutamic, edetic, stearic, palmitic, oleic, lauric, pantothenic, tannic, ascorbic and valeric acids.

By “pharmaceutically acceptable derivative” is meant any pharmaceutically acceptable salt, hydrate, solvate or any other compound which, upon administration to the subject, is capable of providing (directly or indirectly) a compound described herein and/or an active metabolite or residue thereof.

The term “pro-drug” is used herein in its broadest sense to include those compounds which are converted in vivo to a compound described herein.

“Alkyl” refers to monovalent saturated aliphatic hydrocarbyl groups having from 1 to 10 carbon atoms and, in some embodiments, from 1 to 6 carbon atoms. “Cₓ-C_{y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Substituted imine” refers to an imine group having from 1 to 5 and, in some embodiments, 1 to 3 or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene or alkylene groups include branched and straight chain hydrocarbyl groups. For example “(C₁₋₆)alkylene” is meant to include methylene, ethylene, propylene, 2-methypropylene, pentylene, and the like.

The term “aralkyl” refers to the term aryl-alkylene wherein alkylene is as defined above and aryl is as defined below. Examples of this group include, but are not limited to benzyl, phenethyl, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy” refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, sulfur, S(O), S(O)₂, and NR₁₁ and includes single ring (e.g. imidazolyl) and multiple ring systems (e.g. benzimidazol-2-yl and benzimidazol-6-yl). NR₁₁ is selected from hydrogen and C₁-C₆ alkyl. For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, benzothienyl, or oxindolyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated or unsaturated cyclic group having from 1 to 10 carbon atoms and from 1 to 4 heteroatoms selected from the group consisting of oxygen, nitrogen, sulfur, S(O), S(O)₂, and NR₁₁ and includes single ring and multiple ring systems including fused, bridged, and spiro ring systems. NR₁₁ is selected from hydrogen and C₁-C₆ alkyl. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocyclyl wherein heterocyclyl is as defined herein.

“Substituted heterocyclyloxy” refers to the group —O-(substituted heterocyclyl) wherein substituted heterocyclyl is as defined herein.

“Heterocyclylthio” refers to the group —S-heterocyclyl wherein heterocyclyl is as defined herein.

“Substituted heterocyclylthio” refers to the group —S-(substituted heterocyclyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiornorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

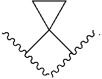

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

Terms not defined herein have their commonly accepted meaning that is well understood by the skilled artisan.

### Descriptive Embodiments

One aspect of this invention relates to a treatment of a viral infection in a subject or patient caused by enterovirus, parechovirus, or other virus classes using one or more of the small molecules provided herein. Furthermore, also disclosed herein is the use of said small molecules for in vitro assays. For determining anti-viral activity according to this invention, the quantification of virus replication, cell death and/or cell viability can be used as a surrogate and measured by a variety of assays known in the art. These include, for example, live cell metabolic assays such as commercial kits ATPlite™ assay, CellTiter-Glo® assay, cytoloysis or membrane leakage assays such as propidium iodide, trypan blue, and 7-Aminoactinomycin D assays; mitochondrial activity or caspase assays such as resazurin and formazan assays; functional assays that assay for cell activity, plaque reduction assays, and genomic and proteomic assays that assay for activation of stress pathways using DNA microarrays and protein chips.

In one embodiment, the progress of viral infection is measured by a plaque reduction assay. In the plaque assay, each infectious virus particle multiplies under conditions that result in a localized area of infected cells or ‘plaque’. The plaques are revealed either as areas of dead/destroyed cells by the virus detected by general cellular stains or as areas of infected cells detected by immuno-staining. In one embodiment, the Log 10 reduction is greater than about 1. In another embodiment the Log 10 reduction is greater than about 3. In a further embodiment, the Log 10 reduction is greater than about 3.5. The Log 10 reduction is measured as log₁₀ (X/Y) where X is equal to the viral titer without compound and Y is equal to viral titer with the compound.

A further aspect relates to a method for reducing the microbial load of enterovirus in a cell infected with enterovirus by contacting the cell or administering to a subject in need thereof a compound as disclosed herein and identified for such purpose. In some embodiments, provided herein is a method for reducing the microbial load of enterovirus in a cell infected with enterovirus or other virus, said method comprising contacting the cell with an effective amount of a compound selected from the group consisting of a compound of Formula I, Formula II, Formula IIIa, Formula IIIb, Formula IV, or a compound of Table 1. A further aspect relates to a method for reducing the microbial load of parechovirus in a cell infected with parechovirus by contacting the cell or administering to a subject in need thereof a compound as disclosed herein and identified for such purpose. As used herein, the microbial load refers to the number of organisms, enterovirus or parechovirus, for example, contaminating an object, such as a cell. The Log 10 reduction is measured as log₁₀ (X/Y) where X is equal to viral RNA copies of an RNA molecule without compound and Y is equal to viral RNA copies of an RNA molecule with the compound. In a certain embodiment, the Log 10 reduction is greater than about 1.5. In a further embodiment, the Log 10 reduction is greater than about 4. In other embodiments, the Log 10 reduction is greater than about 0.2, 2, 3, 3.5, 4, 4.5, or 5. In some embodiments, the contacting of the cell is performed in vitro. In some embodiments, the contacting of the cell is performed in vivo.

Another aspect relates to a method for inhibiting the replication of enterovirus in a cell infected with enterovirus, said method comprising contacting the cell with an effective amount of a compound described herein. Another aspect relates to a method for inhibiting the replication of parechovirus in a cell infected with parechovirus, said method comprising contacting the cell with an effective amount of a compound described herein. Replication of virus can be measured by methods known in the art. These methods include, for example, reverse transcriptase PCR (RT-PCR) methods that assay for viral RNA, plaque assays, fluorescent focus assay, and assays that quantify viral protein production. In one embodiment, the assay used is RT-PCR. In some embodiments, the contacting of the cell is performed in vitro. In some embodiments, the contacting of the cell is performed in vivo.

Another aspect provides a method for treating a viral infection, said method comprising administering to a patient with a viral infection an effective amount of a compound selected from the group consisting of a compound of Formula I, Formula II, Formula IIIa, Formula IIIb, Formula IV, or a compound of Table 1.

Another aspect relates to a method for treating an enteroviral infection, said method comprising administering to a patient with an enteroviral infection an effective amount of one or more compound(s) described herein. An effective amount is one readily determined by methods described herein and by methods known to those skilled in the art. Enteroviral infections include, for example, infections of echovirus 9, echovirus 11, echovirus 30, Coxsackievirus B5, Echovirus 6, Coxsackievirus B2, Coxsackievirus A9, Echovirus 4, Coxsackievirus B4, Echovirus 7, Coxsackievirus B3, Echovirus 18, Coxsackievirus B1, Echovirus 3, Echovirus 5, Echovirus 13, Coxsackievirus A16, Coxsackievirus A24, Enterovirus 68, Enterovirus 71. Human Parechoviruses 1 and 2 are also considered, as they were originally taxonomically classified as enteroviruses. It is contemplated that compounds described herein can be used to treat infections in subjects infected with enteroviruses, such as, for example, the aforementioned enteroviruses. It is further contemplated that the that compounds described herein can be used to treat the clinical manifestations of enteroviral infections such as, for example, aseptic meningitis, meningoencephalitis, myopericarditis, encephalomyocarditis syndrome, acute flaccid paralysis, gastrointestinal illnesses, myocarditis, neurologic illnesses (e.g., aseptic meningitis, meningoencephalitis, and encephalitis), febrile rash illnesses, respiratory illnesses, paralysis, neonatal systemic illness, meningoencephalitis in immunodeficient persons, herpangina, rash illnesses, neonatal sepsis, pleurodynia hand, foot, and mouth disease, chronic infection of immunodeficient hosts, fatal myocarditis, rhabdomyolysis with renal failure, neonatal febrile illness, acute hemorrhagic conjunctivitis, polio-like paralysis, bulbar encephalitis encephalomyelitis, and neonatal sepsis with necrotizing enterocolitis. Furthermore, we anticipate that the aforementioned compounds might be as well useful for the treatment of other viral infections, including the larger family of picornaviruses. Furthermore, in accordance with this invention, it is contemplated that compounds utilized herein are active against members of the parechovirus and hepatovirus genera. In some embodiments, it is contemplated that the compounds utilized herein are useful for treatment of hepatitis A infection,

A significant application for the compounds described herein is for use in humans with an enteroviral infection. However, the compounds can also be used to offer protection against an enteroviral infection in non-human subjects.

In another aspect, provided herein is a method for inhibiting the viral cell entry, budding of enterovirus and/or other viruses in a cell infected with said virus, said method comprising contacting the cell with an effective amount of a compound selected from the group consisting of a compound of Formula I, Formula II, Formula IIIa, Formula IIIb, Formula IV, or a compound of Table 1. In some embodiments, the virus is a parechovirus. In some embodiments, the contacting of the cell is performed in vitro. In some embodiments, the contacting of the cell is performed in vivo.

The foregoing uses are illustrative and not limiting. Using the teaching provided herein, other uses of the anti-enteroviral agents described herein will be readily available to one of skill in the art and might be as well applied to other viral infections. In some aspect, the methods are performed by contacting or administering only the compounds as disclosed herein and therefore would exclude compounds that may exert their effect by a different mechanism of action or pathway. In those aspect, the methods would consist essentially or consist of administration or contacting with the compound or compounds of interest.

### Compounds

I. Pyrazolo[3,4-b]pyridine compounds and derivative(s)

It was discovered that pyrazolo[3,4-b]pyridine compounds have potent antiviral activities. Examples of pyrazolo[3,4-b]pyridine are listed in Table 1. Accordingly, in certain embodiments, the compound administered in methods described herein are pyrazolo[3,4-b]pyridine, salts (e.g., pharmaceutically acceptable salts) thereof and/or solvates thereof. In certain embodiments, the administered compound is a pyrazolo[3,4-b]pyridine according to Formula I:

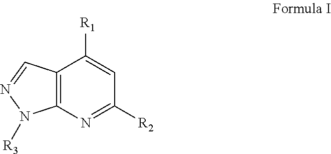

wherein R₁ is defined by the structure below:

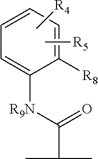

R₄ and R₅ are independently selected from the group consisting of hydrogen, halo, C₁-C₆ alkyl, C₁-C₆ haloalkyl, C₁-C₆ alkoxy, heteroaryl, heterocycle, C₆-C₁₄ aryl, C₃-C₈ cycloalkyl, —SO₂(R₆), —CN, and C₂-C₆ alkenyl, wherein heteroaryl, heterocycle, cycloalkyl, and aryl is optionally substituted with 1-4 R₇ groups or

R₄ and R₅ are linked as R₄+R₅;

R₂ is selected from the group consisting of heterocycle, heteroaryl, C₃-C₈ cycloalkyl, C₆-C₁₄ aryl, and C₁-C₆ alkyl;

R₃ is C₁-C₆ alkyl;

R₆ is selected from the group consisting of C₁-C₆ alkyl, substituted C₁-C₆ alkyl, NHR₁₀, N(R₁₀)₂ wherein each R₁₀ can be joined with the nitrogen atom to form a heterocyclic or a heteroaryl;

R₇ is selected from the group consisting of C₁-C₆ alkyl, carbonyl, and sulfonyl;

R₈ is hydrogen or C₁-C₆ alkyl;

R₉ is selected from the group consisting of hydrogen and C₁-C₆ alkyl; and

R₁₀ is selected from the group consisting of hydrogen and C₁-C₆ alkyl.

In addition, salts thereof are contemplated.

In addition, pro-drug forms of Formula I and other optimally substituted compounds of this class are contemplated.

In certain embodiments, each R₄ and R₅ are independently selected from the group consisting of: F; methyl; isopropyl;

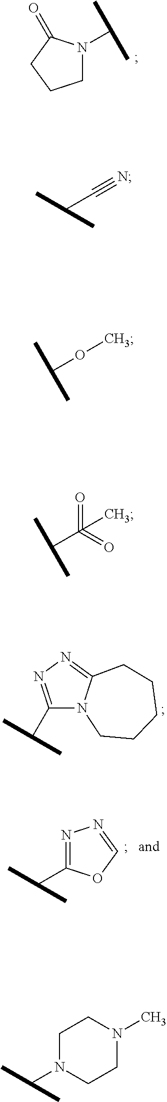

In certain embodiments, when R₄ and R₅ are linked as R₄+R₅ and R₄+R₅ is selected from the group consisting of

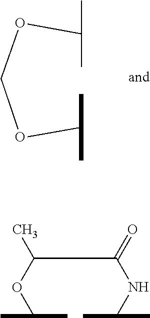

In certain embodiments, R₂ is selected from the group consisting of:

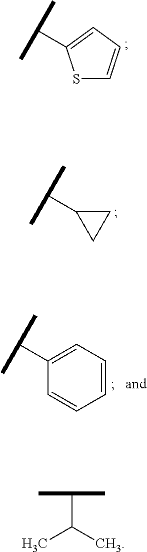

In certain embodiments, R₃ is selected from the group consisting of

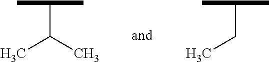

In certain embodiments, R₈ is hydrogen, and R⁴ and R⁵ are non-hydrogen substituents. In certain embodiments, R₈ is hydrogen, and one of R₄ and R₅ are non-hydrogen substituents. In certain embodiments, R₈ is methyl and R₉ is hydrogen.

In certain embodiments, R₁ is selected from the group consisting of:

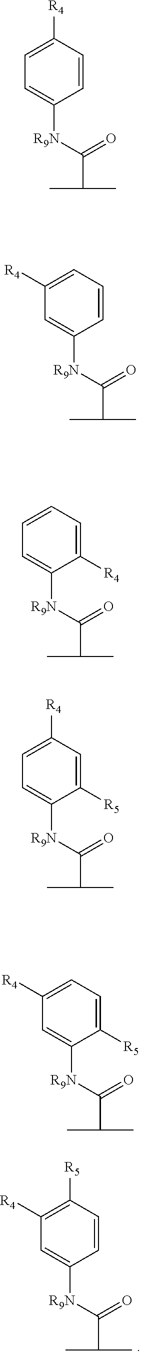

### II. Quinoline Compounds and Derivative(s)

It was discovered that quinoline compounds have potent antiviral activities. Examples of quinoline compounds are listed in Table 1. Accordingly, in certain embodiments, the administered compound is quinoline, salts (e.g., pharmaceutically acceptable salts) thereof and/or solvates thereof. In certain embodiments, the compound is a quinoline according to Formula II:

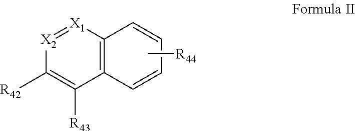

X₁ and X₂ are independently selected from N, CH, and C(R₄₁) wherein at least one of X₁ and X₂ is N;

R₄₁, R₄₂, and R₄₃ are independently selected from hydrogen, halo, hydroxy, C₁-C₆ alkyl, substituted acyl, CH₂N₃, CN, C₁-C₆ alkoxy, C₁-C₆ haloalkyl, C₆-C₁₄ aryl, acetyl, C₃-C₈ cycloalkyl, heterocycle, and heteroaryl wherein said C₁-C₆ alkyl, heterocycle, heteroaryl, and C₆-C₁₄ aryl is optionally substituted with 1-4 R₄₉ groups;

R₄₄ is selected from the group consisting of hydrogen, halo, C₁-C₆ alkyl, C₆-C₁₄ aryl, acetyl, and C₁-C₆ haloalkyl;

R₄₉ is selected from the group consisting of hydrogen, hydroxyl, heterocycle, C₁-C₆ alkyl, halo, carbonyl, sulfonyl, heteroaryl, substituted heteroaryl, substituted heterocycle, NH(R₄₈), NH(R₄₈)₂, and substituted C₁-C₆ alkyl or

wherein R₄₇ is selected from the group consisting of hydrogen, substituted imine, NHR₅₀, N(R₅₀)₂, substituted C₆-C₁₄ aryl, substituted heterocycle, substituted heteroaryl, and substituted C₁-C₆ alkyl; and

R₅₀ is C₁-C₆ alkyl.

In one embodiment, X₁ is N and X₂ is CH. In a further embodiment, X₁ is N and X₂ is C(R₄₁).

In certain embodiments, R₄₁, R₄₂, and R₄₃ are independently selected from the group consisting of hydrogen, F, Cl, Br, I, methyl, CH₂N₃, CF₃, phenyl, acetyl, or R₄₁, R₄₂, and R₄₃ are independently selected from the group consisting of

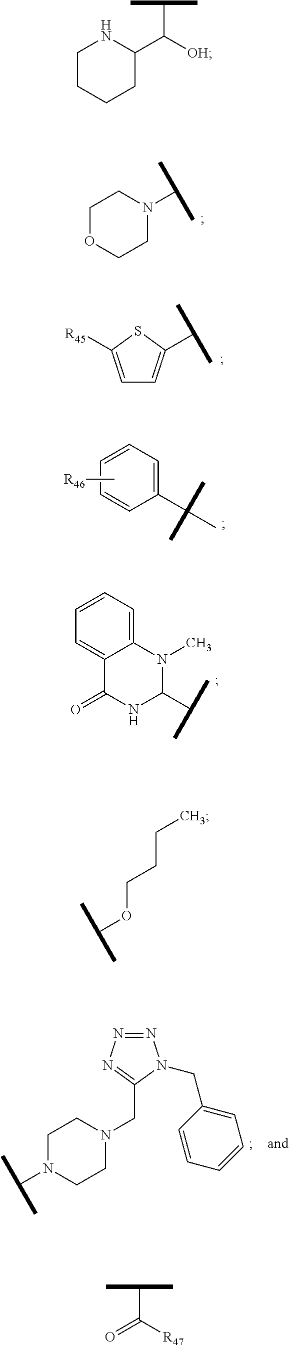

wherein R₄₅ and R₄₆, are independently selected from the group consisting of hydrogen, halo, and C₁-C₆ alkyl and R₄₇ is methyl or NH(R₄₈) wherein R₄₈ is as previously defined.

In further embodiments, R₄₈ is selected from the group consisting of

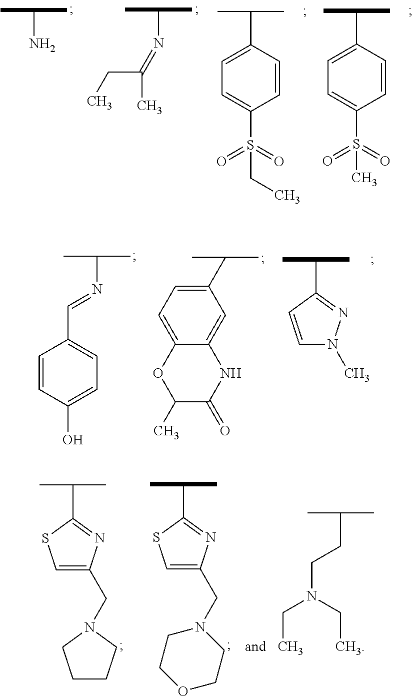

In addition, tautomers, salts and/or solvates, and/or esters thereof are contemplated.

In addition, other quinoline derivatives with antiviral activity are contemplated. In certain embodiments, a quinoline derivative according to the structure below is contemplated:

### N-[(7,8-Dimethyl-2-oxo-1,2-dihydro-3-quinolinyl)methyl]-N-(4-fluorobenzyl)-2-thiophenecarboxamide

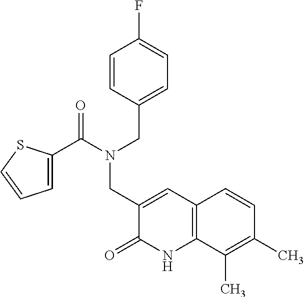 [SMILES: Cc1ccc2cc(CN(Cc3ccc(F)cc3)C(=O)c3cccs3)c(=O)[nH]c2c1C]

### N-[3-(2-Methyl-1,3-thiazol-4-yl)phenyl]-2-oxo-1,2,3,4-tetrahydro-6-quinolinesulfonamide

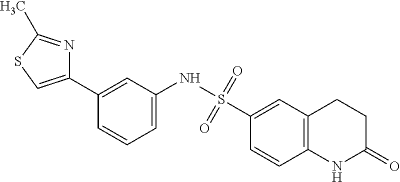 [SMILES: Cc1nc(-c2cccc(NS(=O)(=O)c3ccc4c(c3)CCC(=O)N4)c2)cs1]

In addition, prodrug forms of a quinoline and other optimally substituted compounds of this class are contemplated.

### III. Benzimidazole Compounds and Derivative(s)

It was discovered that benzimidazole compounds have potent antiviral activities. Examples of benzimidazole are listed in Table 1. Accordingly, in certain embodiments, the compound is a benzimidazole, salts (e.g., pharmaceutically acceptable salts) thereof and/or solvates thereof. In certain embodiments, the compound is a compound of Formula IIIa or IIIb:

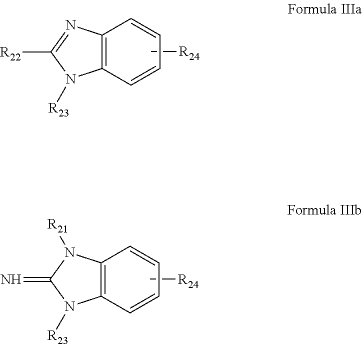

wherein R₂₁, R₂₂, R₂₃, and R₂₄ are independently selected from the group consisting of C₁-C₆ alkoxy and C₁-C₆ alkyl optionally substituted with 1-4R₂₆;

R₂₆ is selected from the group consisting of C₆-C₁₄ aryl, substituted C₆-C₁₄ aryl, C(O)R₂₇; S(R₂₇); and heterocycle;

R₂₇ is selected from the group consisting of substituted C₆-C₁₄ aryl and substituted heteroaryl.

In addition, salts and/or solvates, and/or esters thereof are contemplated.

In addition, prodrug forms of a benzimidazole and other optimally substituted compounds of this class are contemplated.

In certain embodiments, R₂₁, R₂₂, R₂₃, and R₂₄ are independently selected from the group consisting of

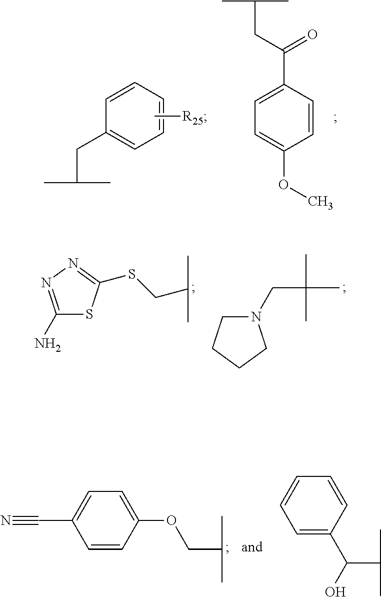

wherein R₂₅ is hydrogen or methyl.

### IV. 1,4-Benzodioxan or 1,2-Ethylenedioxybenzene Compounds and Derivative(s)

It was discovered that 1,4-benzodioxan or 1,2-ethylenedioxybenzene compounds have potent antiviral activities. Examples of 1,4-Benzodioxan or 1,2-Ethylenedioxybenzene compounds are listed in Table 1. Accordingly, in certain embodiments, the administered compound is 1,4-benzodioxan or 1,2-ethylenedioxybenzene, salts (e.g., pharmaceutically acceptable salts) thereof and/or solvates thereof are contemplated. In certain embodiments, the compound is a compound according to Formula IV:

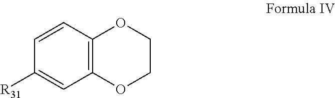

wherein R₃₁ is of the formula:

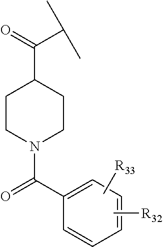

wherein R₃₂ and R₃₃ are independently hydrogen, halo, C₁-C₆alkyl, C₁-C₆ haloalkyl, C₁-C₆ alkoxy, and heteroaryl.

In certain embodiments, R₃₂ and R₃₃ are independently selected from the group consisting of hydrogen, F, Cl, Br, I, methyl, CF₃,

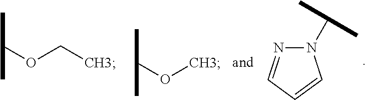

In certain embodiments, at least one of R₃₂ and R₃₃ is a non-hydrogen substituent. In certain embodiments, both of R₃₂ and R₃₃ are non-hydrogen substituents. In certain embodiments, R₃₁ is one of formula:

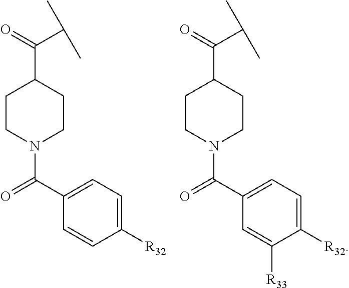

In addition, salts and/or solvates, and/or esters thereof are contemplated.

In addition, prodrug forms of Formula IV are also contemplated.

### V. Additional Antiviral Agents

In addition, to the above compounds of the formula 1-4 described herein and their derivatives it was also discovered that a number of other agents offer similar antiviral activities and are useful in the methods disclosed herein. Such agents and other optimally substituted compounds of this class include, but are not limited to, the following:

### 1. Quinazolinones

### 3-Benzyl-2-(propylsulfanyl)-4(3H)-quinazolinone and: 3-Benzyl-2-(2-propyn-1-ylsulfanyl)-4(3H)-quinazolinone

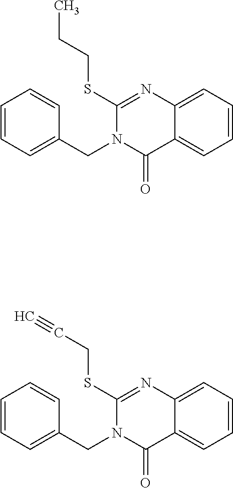 [SMILES: CCCSc1nc2ccccc2c(=O)n1Cc1ccccc1 ; C#CCSc1nc2ccccc2c(=O)n1Cc1ccccc1]

### N-(2-Methoxy-6-methylindolo[1,2-c]quinazolin-12-yl)acetamide and N-(6-Methylindolo[1,2-c]quinazolin-12-yl)-2-pyrazinecarboxamide

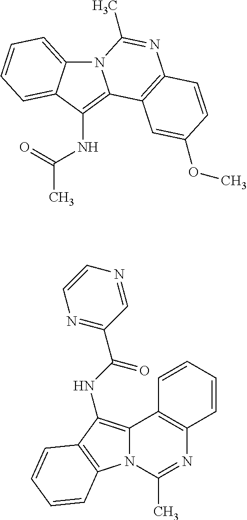 [SMILES: COc1ccc2nc(C)n3c4ccccc4c(NC(C)=O)c3c2c1 ; Cc1nc2ccccc2c2c(NC(=O)c3cnccn3)c3ccccc3n12]

### N′-[(11Z)-11H-Indeno[1,2-b]quinoxalin-11-ylidene]nicotinohydrazide

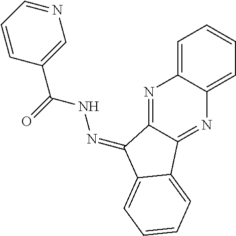 [SMILES: O=C(N/N=C1/c2ccccc2-c2nc3ccccc3nc21)c1cccnc1]

### 2. Pyrimidines

### 2-({4-[2-(4-Methoxyphenyl)ethyl]-1-piperidinyl}methyl)-4H-pyrido[1,2-a]pyrimidin-4-one

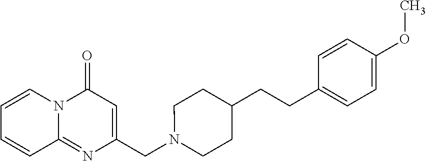 [SMILES: COc1ccc(CCC2CCN(Cc3cc(=O)n4ccccc4n3)CC2)cc1]

### 5-(4-Bromophenyl)-4-[(5-cyclopropyl-1,3,4-oxadiazol-2-yl)sulfanyl]thieno[2,3-d]pyrimidine

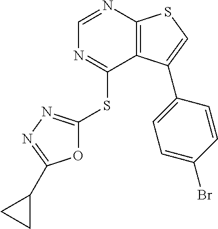 [SMILES: Brc1ccc(-c2csc3ncnc(Sc4nnc(C5CC5)o4)c23)cc1]

### 3-[2-(1,3-Benzodioxol-5-yl)-2-oxoethyl]-5-(3,4-dimethoxyphenyl)thieno[2,3-d]pyrimidin-4(3H)-one

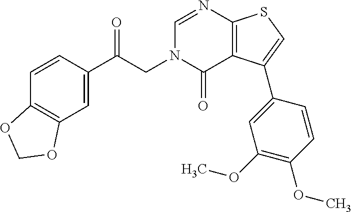 [SMILES: COc1ccc(-c2csc3ncn(CC(=O)c4ccc5c(c4)OCO5)c(=O)c23)cc1OC]

### 3. Benzoic Acids

### 3-{[(4-Methylphenyl)sulfinyl]amino}benzoic acid and 3-[(4-Biphenylylsulfonyl)amino]benzoic acid

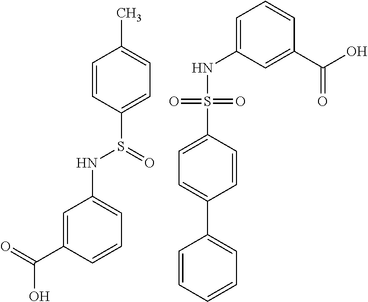

### 4. Furamides

### N-(4-Fluorobenzyl)-N-(4-methoxyphenyl)-2-furamide and N-Benzyl-N-(2-pyridinylmethyl)-2-furamide

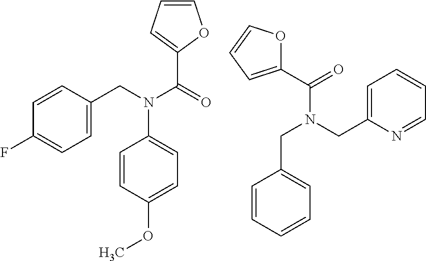

### 5. Carboxamide

### 2-(4-Methoxyphenyl)-4-methyl-N-[3-(6,7,8,9-tetrahydro-5H-[1,2,4]triazolo[4,3-a]azepin-3-yl)phenyl]-1,3-thiazole-5-carboxamide

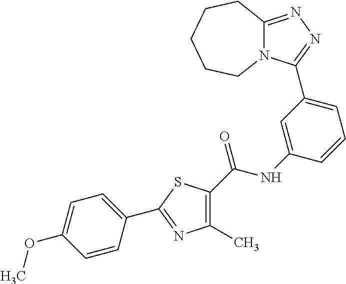 [SMILES: COc1ccc(-c2nc(C)c(C(=O)Nc3cccc(-c4nnc5n4CCCCC5)c3)s2)cc1]

### (2Z)-2-[(4-Methoxyphenyl)imino]-N-(tetrahydro-2-furanylmethyl)-2H-chromene-3-carboxamide

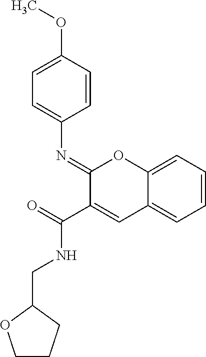 [SMILES: COc1ccc(/N=c2\oc3ccccc3cc2C(=O)NCC2CCCO2)cc1]

### 1-{2-Oxo-2-[(7E)-3-(2-thienyl)-7-(2-thienylmethylene)-3,3a,4,5,6,7-hexahydro-2H-indazol-2-yl]ethyl}-4-piperidinecarboxamide

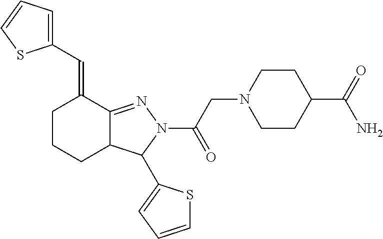 [SMILES: NC(=O)C1CCN(CC(=O)N2N=C3/C(=C/c4cccs4)CCCC3C2c2cccs2)CC1]

### 3-Benzyl-N-(6-methoxy-3-pyridinyl)-4-oxo-3,4-dihydro-1-phthalazinecarboxamide

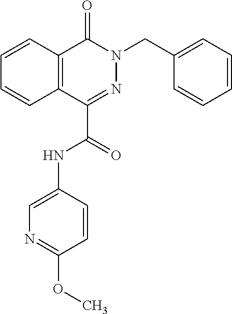 [SMILES: COc1ccc(NC(=O)c2nn(Cc3ccccc3)c(=O)c3ccccc23)cn1]

### 6. Imidazo[1,2-a]pyrazines

### (2-Methoxy-5-{3-[(4-methylbenzyl)amino]imidazo[1,2-a]pyrazin-2-yl}phenyl)methanol and 2-[4-Methoxy-3-(4-morpholinylmethyl)phenyl]-N-(2-methyl-2-butanyl)imidazo[1,2-a]pyrazin-3-amine

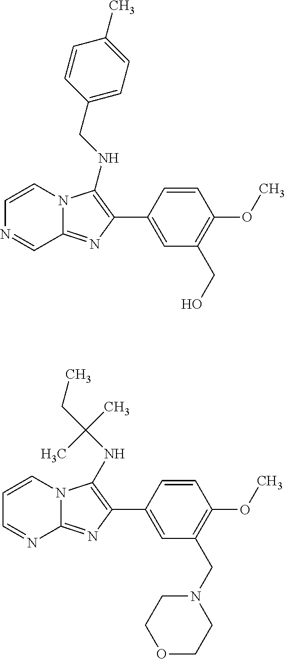 [SMILES: COc1ccc(-c2nc3cnccn3c2NCc2ccc(C)cc2)cc1CO ; CCC(C)(C)Nc1c(-c2ccc(OC)c(CN3CCOCC3)c2)nc2ncccn12]

### N²-Butyl-N-[2-(1-piperidinyl)phenyl]glycinamide

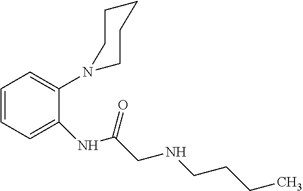 [SMILES: CCCCNCC(=O)Nc1ccccc1N1CCCCC1]

### N,N-Diethyl-2-[(6-methyl-2,3,4,9-tetrahydro-1H-carbazol-1-yl)amino]ethanaminium chloride

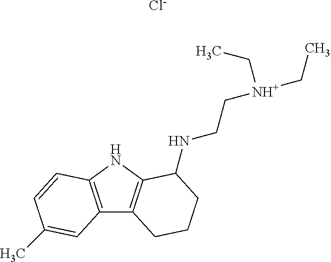 [SMILES: [Cl-] ; CC[NH+](CC)CCNC1CCCc2c1[nH]c1ccc(C)cc21]

### 4-(2-Naphthyl)-1,4-dihydro[1,3,5]triazino[1,2-a]benzimidazol-2-amine

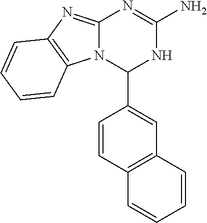 [SMILES: NC1=Nc2nc3ccccc3n2C(c2ccc3ccccc3c2)N1]

### 4-[(1E)-1-Phenyl-1-propen-2-yl]-1,4-dihydro[1,3,5]triazino[1,2-a]benzimidazol-2-amine

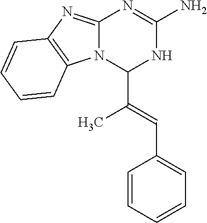 [SMILES: C/C(=C\c1ccccc1)C1NC(N)=Nc2nc3ccccc3n21]

### 1-Cyclohexyl-4-[4-(3-phenyl-1,2,4-oxadiazol-5-yl)butanoyl]-2-piperazinone

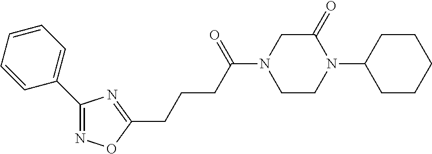 [SMILES: O=C(CCCc1nc(-c2ccccc2)no1)N1CCN(C2CCCCC2)C(=O)C1]

### Flupentixol: 2-(4-{(3Z)-3-[2-(Trifluoromethyl)-9H-thioxanthen-9-ylidene]propyl}-1-piperazinyl)ethanol

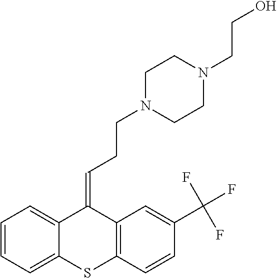 [SMILES: OCCN1CCN(CC/C=C2/c3ccccc3Sc3ccc(C(F)(F)F)cc32)CC1]

### Zuclopenthixol: 2-{4-[(3Z)-3-(2-Chloro-9H-thioxanthen-9-ylidene)propyl]-1-piperazinyl}ethanol

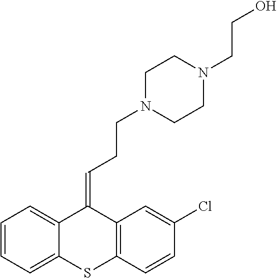 [SMILES: OCCN1CCN(CC/C=C2/c3ccccc3Sc3ccc(Cl)cc32)CC1]

### Fluoxetine: N-Methyl-3-phenyl-3-[4-(trifluoromethyl)phenoxy]-1-propanamine

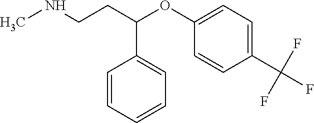 [SMILES: CNCCC(Oc1ccc(C(F)(F)F)cc1)c1ccccc1]

### Lycorine: (1S,2S,12bS,12cS)-2,4,5,7,12b, 12c-Hexahydro-1H-[1,3]dioxolo[4,5-j]pyrrolo[3,2,1-de]phenanthridine-1,2-diol

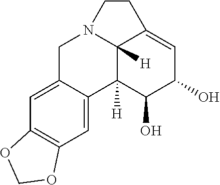

It is contemplated that one or more of these agents can be formulated and used in a manner analogous to fluoxetine and other optimally substituted compounds of this class.

### Pharmaceutical Formulations

In certain embodiments one or more active agents described herein (e.g., fluoxetine, fluoxetine derivative(s), and/or other antiviral agents described herein) are administered to a mammal in need thereof, e.g., to a mammal infected with infectious virus (e.g. an enterovirus)

The active agent(s) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience. For example, PCT Publication No: WO 2000/059863 teaches the formulation of disodium salts, monohydrates, and ethanol solvates of a variety of delivery agents.

Similarly, acid salts of active agents (e.g., the therapeutic and/or prophylactic agents described herein) can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agent(s) identified herein can be administered in a number of ways depending upon whether local or systemic treatment is desired and upon the area to be treated. The agent(s) are useful for parenteral, topical (including ophthalmic), to mucus membranes (including vaginal and rectal delivery), pulmonary (e.g. by inhalation or insufflation of powders or aerosols, including by nebulizer), intratracheal, intranasal, epidermal, transdermal, oral, nasal, subcutaneous, intramuscular, intravenous, or local administration, such as by, for prophylactic and/or therapeutic treatment for conditions or symptoms caused by infectious viruses (e.g. an enterovirus).

The active agents described herein also be combined with a pharmaceutically acceptable carrier and/or excipient to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., Fluoxetine, Fluoxetine derivative(s), and/or other antiviral agents described herein,) and the resulting composition is compressed. Where necessary the compressed product is coated, e.g., for masking the taste or for enteric dissolution or sustained release. Suitable coating materials include, but are not limited to, ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets, capsules, gelcaps, and the like, sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising one or more active agent(s) (e.g., agents described herein) herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

For topical administration the active agent(s) described herein can be formulated as solutions, gels, ointments, creams, suspensions, and the like as are well-known in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can optionally contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal or other transepithelial administrations, penetrants appropriate to the barrier to be permeated can be used in the formulation. Such penetrants are generally known in the art.

For oral administration, the compounds can be readily formulated by combining the active agent(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the active agent(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver one or more active agent(s) described herein. Certain organic solvents such as dimethylsulfoxide also can be employed, although usually at the cost of greater toxicity. Additionally, the compounds may be delivered using a sustained-release system, such as semipermeable matrices of solid polymers containing the therapeutic/prophylactic agent(s). Various uses of sustained-release materials have been established and are well known by those skilled in the art. Sustained-release capsules may, depending on their chemical nature, release the compounds for a few days to a few weeks to up to over 100 days. Depending on the chemical nature and the biological stability of the active agent(s), additional strategies for stabilization may be employed.

In certain embodiments, the active agent(s) described herein are administered to the oral cavity. This is readily accomplished by the use of lozenges, aersol sprays, mouthwash, coated swabs, and the like.

In certain embodiments, the active agent(s) of this invention are administered topically, e.g., to the skin surface, to a surgical site, and the like.

In certain embodiments the active agents of this invention are administered systemically (e.g., orally, or as an injectable) in accordance with standard methods well known to those of skill in the art. In other embodiments, the agents, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active agent(s)” that is ultimately available for delivery to the surface of the skin. Thus, for example, the “reservoir” may include the active agent(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While pharmacological formulation and administration is described with respect to use in humans, it is also suitable for animal, e.g., veterinary use. Thus certain preferred organisms include, but are not limited to humans, non-human primates, canines, equines, felines, porcines, ungulates, lagomorphs, and the like.

In certain embodiments prodrug and/or extended release formulations of the antiviral agents described herein are contemplated.

In certain embodiments, the use of polymeric drug delivery system is contemplated. Controlled drug delivery occurs when a polymer, whether natural or synthetic, is combined with one or more active antiviral agents described herein in such a way that the active agent(s) are released from the material in a predesigned manner. The release of the active agent(s) may be constant over a long period, it may be cyclic over a long period, or it may be triggered by the environment or other external events. In particular the use of controlled-delivery systems can result in the maintenance of drug levels within a desired range, the need for fewer administrations, optimal use of the drug in question, and increased patient compliance.

A wide range of materials have been employed to control the release of drugs and other active agents and the use of these materials with the antiviral agents described herein is contemplated. Some suitable materials include but are not limited to poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, and polyorthoesters. There are three primary mechanisms by which active agents can be released from a delivery system: diffusion, degradation, and swelling followed by diffusion. Any or all of these mechanisms may occur in a given release system. Diffusion occurs when a drug or other active agent passes through the polymer that forms the controlled-release device. The diffusion can occur on a macroscopic scale—as through pores in the polymer matrix—or on a molecular level, by passing between polymer chains.

Other polymeric delivery system are known to those of skill in the art. For example, U.S. Pat. No. 5,942,252 describes a microcapsule comprising as its biocompatible excipient a poly(lactide-co-glycolide), poly(lactide), poly(glycolide), copolyoxalate, polycaprolactone, poly(lactide-co-caprolactone), poly(esteramide), polyorthoester, poly(p-hydroxybutyric) acid and/or polyanhydride for use in delivering agents into and through mucosally-associated lymphoid tissue.

PCT Publication WO 98/36013 describes aliphatic-aromatic dihydroxy compounds for use as controlled drug delivery systems. PCT Publication WO 97/39738 describes preparation of microparticles of a sustained release ionic conjugate comprising a free carboxyl group containing biodegradable polymers and a free amino group-containing drug. PCT Publication WO 02/09768 discloses [polymers (i.e. polyesters, polyamides, and polythioesters or a mixture thereof) that comprise active agent(s) and degrade hydrolytically into the biologically active agents.

In certain embodiments the use of nanoparticle formulations is contemplated. For drug delivery not only engineered particles may be used as carrier, but also the drug itself may be formulated at a nanoscale, and then function as its own “carrier”. The composition of the engineered nanoparticles may vary. Source materials may be of biological origin like phospholipids, lipids, lactic acid, dextran, chitosan, or have more “chemical” characteristics like various polymers (e.g., the polymers described above), carbon, silica, and metals.

Other suitable prodrug formulations include, for example, the use of amino, or otherwise modified, derivatives of the active agents described herein. In this regard, it is noted that U.S. Patent publication No: 20060287283 teaches prodrugs of 9-aminomethyltetracycline compounds and it is contemplated that the active agents described herein can be similarly modified.

### Effective Dosages

The active agents described herein (e.g., agents described herein) will generally be used in an amount effective to achieve the intended purpose (e.g., to reduce, repair, or prevent damage to cells, tissues, or organs induced by infection). Of course, it is to be understood that the amount used will depend on the particular application. By therapeutically effective amount is meant an amount of active agent or composition comprising such that inhibitor eliminates infection induced symptoms or the progression of infection-induced damage to cells, tissues, or organs or that aids in the reversal of infection-induced damage to cells, tissues, or organs. By prophylactically effective amount is meant an amount of active agent or composition comprising such that prevent or reduce infection induced symptoms). An ordinarily skilled artisan will be able to determine effective amounts of particular active agent(s) or combinations thereof for particular applications without undue experimentation using, for example, in vitro or in vivo assays known to those of skill in the art.

In certain therapeutic applications, the compositions of this invention are administered, e.g., topically administered or administered to the oral or nasal cavity, or to a mucosa (e.g., vaginal, pulmonary, rectal, etc.) to a subject suffering from viral infection (clinical or non-clinical) or at risk for viral infection prophylactically to prevent or reduce infection induced symptoms.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compositions of the present invention, and the delivery means, and can generally be estimated based on EC₅₀s found to be effective in in vitro and in vivo animal models.

The dosage/amount of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 150 mg/kg/day. In certain embodiments, dosages range from about 20 mg to about 100 mg given orally twice daily. It will be appreciated that such dosages may be varied to optimize a therapeutic and/or prophylactic regimen in a particular subject or group of subjects. Determination of a therapeutically effective amount is well within the capabilities of those skilled in the art, especially in light of the detailed disclosure provided herein.

For systemic administration, a therapeutically effective dose can be estimated initially from in vitro assays.

Initial dosages can also be estimated from in vivo data, e.g., animal models, using techniques that are well known in the art. One having ordinary skill in the art could readily optimize administration to humans based on animal data. In certain embodiments dosage amount and interval can be adjusted individually to provide plasma levels of the active agent(s) that are sufficient to maintain therapeutic or prophylactic effect.

In cases of local administration or selective uptake, the effective local concentration of active agent(s) may not be related to plasma concentration. One having skill in the art will be able to optimize therapeutically effective local dosages without undue experimentation.

### Safety

Preferably, a therapeutically effective dose of the antiviral agents described herein described herein will provide therapeutic benefit without causing substantial toxicity.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds that exhibit high therapeutic indices are preferred, particularly for in vivo applications. The data obtained from cell culture assays and animal studies can be used in formulating a dosage range that is not toxic for use in human. The dosage of the peptides described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (see, e.g., Fingl et al. (1975) In: The Pharmacological Basis of Therapeutics, Ch. 1, p. 1).

### Kits

In another embodiment this invention provides kits for the inhibition of an infection and/or the inhibition of biofilms (e.g., on a prosthetic or medical implant). The kits typically comprise a container containing one or more of the active agents, e.g., antiviral agents described herein. In certain embodiments the active agent(s) can be provided in a unit dosage formulation (e.g., suppository, tablet, caplet, patch, etc.) and/or may be optionally combined with one or more pharmaceutically acceptable carriers and/or excipients.

In addition, the kits optionally include labeling and/or instructional materials providing directions (i.e., protocols) for the practice of the methods or use of the “therapeutics” or “prophylactics” of this invention. Preferred instructional materials describe the use of one or more active agent(s) of this invention therapeutically or prophylactically to inhibit or prevent infection induced cytopathic effects in vitro or symptoms in vivo. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In certain embodiments of the methods described herein, the compound is selected from the group consisting of the compounds described herein.

In certain embodiments of the methods disclosed herein, the cell is in vitro. In another embodiment, the cell is in vivo.

### EXPERIMENTAL

In the Examples depicted below, the following compounds were tested.

TABLE 1
CHEMICAL COMPOUNDS THAT INHIBIT THE REPLICATION OF
ENTEROVIRUSES AND OTHER PICORNAVIRUSES

| | | | | Antiviral Activity | |
|---|---|---|---|---|---|
| | | | | | In- |
| | | | | Log₁₀ | hibition |
| | | | | reduction | of in- |
| | | Molecular | | of virus | fected |
| Compound | Structure | Formula | Systematic Name | titer ^{a} | cells ^{b} |

Pyrazolo[3,4-b]pyridine

| | | | | | |
|---|---|---|---|---|---|
| 1 | 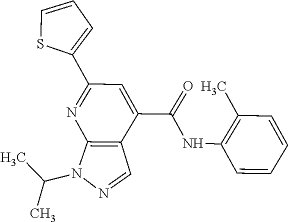 [SMILES: Cc1ccccc1NC(=O)c1cc(-c2cccs2)nc2c1cnn2C(C)C] | C₂₁H₂₀N₄OS | 1-Isopropyl-N-(2- methylphenyl)-6-(2- thienyl)-1H-pyrazolo [3,4-b]pyridine-4- carboxamide | 4.4 | 100% |
| 2 | 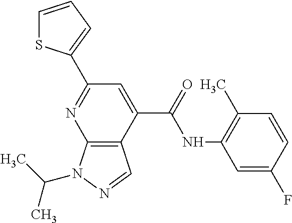 [SMILES: Cc1ccc(F)cc1NC(=O)c1cc(-c2cccs2)nc2c1cnn2C(C)C] | C₂₁H₁₉FN₄OS | N-(5-Fluoro-2- methylphenyl)-1- isopropyl-6- (2-thienyl)-1H- pyrazolo[3,4-b] pyridine-4- carboxamide | 4.4 | 99.6% |
| 3 | 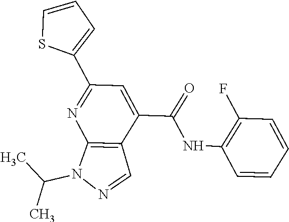 [SMILES: CC(C)n1ncc2c(C(=O)Nc3ccccc3F)cc(-c3cccs3)nc21] | C₂₀H₁₇FN₄OS | N-(2-Fluorophenyl)- 1-isopropyl-6-(2- thienyl)-1H-pyrazolo [3,4-b]pyridine-4- carboxamide | 4.5 | 100% |
| 4 | 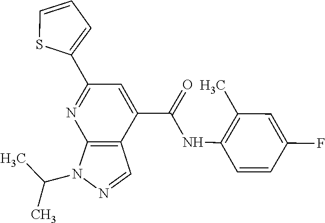 [SMILES: Cc1cc(F)ccc1NC(=O)c1cc(-c2cccs2)nc2c1cnn2C(C)C] | C₂₁H₁₉FN₄OS | N-(4-Fluoro-2- methylphenyl)-1- isopropyl-6- (2-thienyl)-1H- pyrazolo[3,4-b] pyridine-4- carboxamide | 4.5 | 100% |
| 5 | 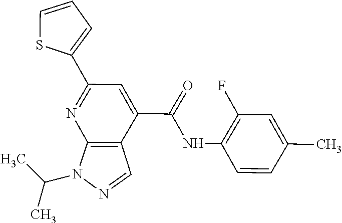 [SMILES: Cc1ccc(NC(=O)c2cc(-c3cccs3)nc3c2cnn3C(C)C)c(F)c1] | C₂₁H₁₉FN₄OS | N-(2-Fluoro-4- methylphenyl)-1- isopropyl-6- (2-thienyl)-1H- pyrazolo[3,4-b] pyridine-4- carboxamide | 4.4 | 99.3% |
| 6 | 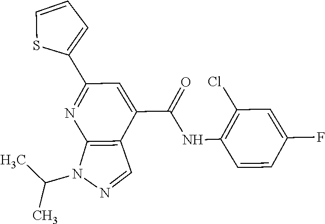 [SMILES: CC(C)n1ncc2c(C(=O)Nc3ccc(F)cc3Cl)cc(-c3cccs3)nc21] | C₂₀H₁₆ClFN₄OS | N-(2-Chloro-4- fluorophenyl)-1- isopropyl-6- (2-thienyl)-1H- pyrazolo[3,4-b] pyridine-4- carboxamide | 4.3 | 99.5% |
| 7 | 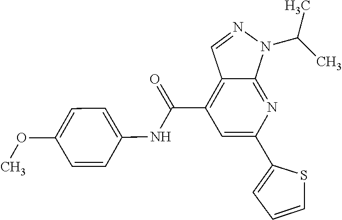 [SMILES: COc1ccc(NC(=O)c2cc(-c3cccs3)nc3c2cnn3C(C)C)cc1] | C₂₁H₂₀N₄O₂S | 1-Isopropyl-N-(4- methoxyphenyl)-6-(2- thienyl)-1H-pyrazolo [3,4-b]pyridine-4- carboxamide | 4.3 | 99.5% |
| 8 | 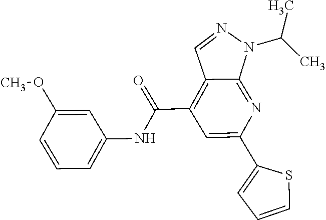 [SMILES: COc1cccc(NC(=O)c2cc(-c3cccs3)nc3c2cnn3C(C)C)c1] | C₂₁H₂₀N₄O₂S | 1-Isopropyl-N-(3- methoxyphenyl)-6-(2- thienyl)-1H-pyrazolo [3,4-b]pyridine-4- carboxamide | 4.4 | 99.2% |
| 9 | 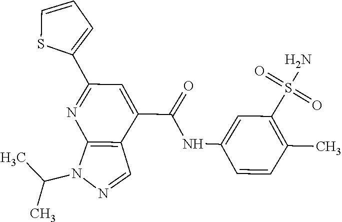 [SMILES: Cc1ccc(NC(=O)c2cc(-c3cccs3)nc3c2cnn3C(C)C)cc1S(N)(=O)=O] | C₂₁H₂₁N₅O₃S₂ | 1-Isopropyl-N- (4-methyl-3- sulfamoylphenyl)- 6-(2-thienyl)-1H- pyrazolo[3,4-b] pyridine-4- carboxamide | 4.5 | 100% |
| 10 | 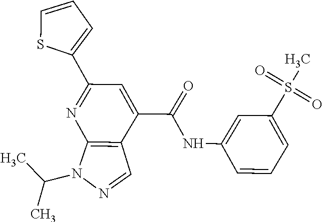 [SMILES: CC(C)n1ncc2c(C(=O)Nc3cccc(S(C)(=O)=O)c3)cc(-c3cccs3)nc21] | C₂₁H₂₀N₄O₃S₂ | 1-lsopropyl-N-[3- (methylsulfonyl) phenyl]-6-(2-thienyl)- 1H-pyrazolo [3,4-b]pyridine-4- carboxamide | 4.4 | 100% |
| 11 | 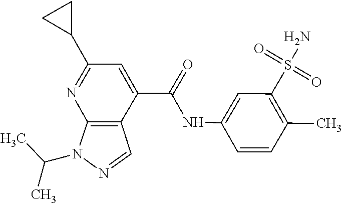 [SMILES: Cc1ccc(NC(=O)c2cc(C3CC3)nc3c2cnn3C(C)C)cc1S(N)(=O)=O] | C₂₀H₂₃N₅O₃S | 6-Cyclopropyl-1- isopropyl-N-(4-methyl- 3-sulfamoylphenyl)- 1H-pyrazolo[3,4- b]pyridine-4- carboxamide | 4.1 | 99.6% |
| 12 | 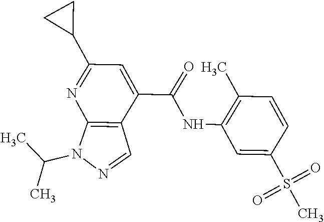 [SMILES: Cc1ccc(S(C)(=O)=O)cc1NC(=O)c1cc(C2CC2)nc2c1cnn2C(C)C] | C₂₁H₂₄N₄O₃S | 6-Cyclopropyl-1- isopropyl-N-[2- methyl- 5-(methylsulfonyl) phenyl]-1H- pyrazolo[3,4-b] pyridine- 4-carboxamide | 4.2 | 100% |
| 13 | 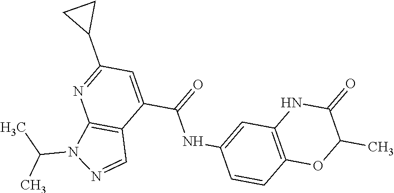 [SMILES: CC1Oc2ccc(NC(=O)c3cc(C4CC4)nc4c3cnn4C(C)C)cc2NC1=O] | C₂₂H₂₃N₅O₃ | 6-Cyclopropyl-1- isopropyl-N- (2-methyl- 3-oxo-3,4-dihydro- 2H-1,4-benzoxazin- 6-yl)-1H-pyrazolo [3,4-b]pyridine-4- carboxamide | 4.2 | 100% |
| 14 | 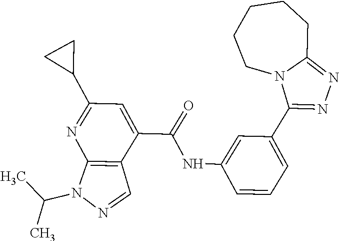 [SMILES: CC(C)n1ncc2c(C(=O)Nc3cccc(-c4nnc5n4CCCCC5)c3)cc(C3CC3)nc21] | C₂₆H₂₉N₇O | 6-Cyclopropyl-1- isopropyl-N-[3- (6,7,8,9- tetrahydro-5H- [1,2,4]triazolo [4,3-a]azepin-3- yl)phenyl]-1H- pyrazolo [3,4-b]pyridine- 4-carboxamide | 3.3 | 100% |
| 15 | 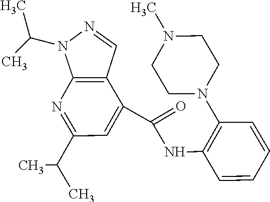 [SMILES: CC(C)c1cc(C(=O)Nc2ccccc2N2CCN(C)CC2)c2cnn(C(C)C)c2n1] | C₂₄H₃₂N₆O | 1,6-Diisopropyl-N- [2-(4-methyl-1- piperazinyl)phenyl)- 1H-pyrazolo[3,4- b]pyridine-4- carboxamide | 2.4 | 100% |
| 16 | 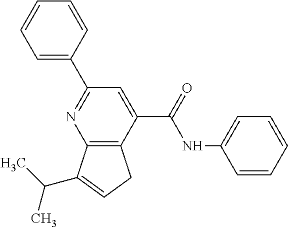 [SMILES: CC(C)C1=CCc2c(C(=O)Nc3ccccc3)cc(-c3ccccc3)nc21] | C₂₂H₂₀N₄O | 1-Isopropyl- N,6-diphenyl-1H- pyrazolo[3,4-b] pyridine- 4-carboxamide | 4.5 | 100% |
| 17 | 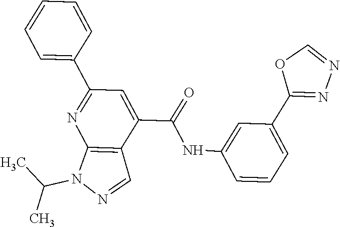 [SMILES: CC(C)n1ncc2c(C(=O)Nc3cccc(-c4nnco4)c3)cc(-c3ccccc3)nc21] | C₂₄H₂₀N₆O₂ | 1-Isopropyl-N-[3- (1,3,4-oxadiazol-2- yl)phenyl]-6-phenyl- 1H-pyrazolo[3,4- b]pyridine-4- carboxamide | 4.3 | 99.6% |
| 18 | 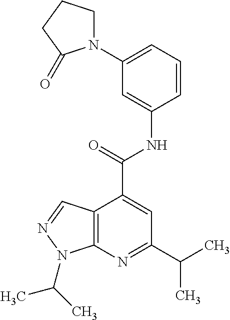 [SMILES: CC(C)c1cc(C(=O)Nc2cccc(N3CCCC3=O)c2)c2cnn(C(C)C)c2n1] | C₂₃H₂₇N₅O₂ | 1,6-Diisopropyl- N-[3-(2-oxo-1- pyrrolidinyl)phenyl]- 1H-pyrazolo[3,4- b]pyridine-4- carboxamide | 4.0 | 97.9% |
| 19 | 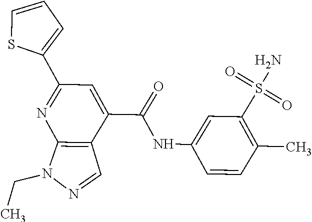 [SMILES: CCn1ncc2c(C(=O)Nc3ccc(C)c(S(N)(=O)=O)c3)cc(-c3cccs3)nc21] | C₂₀H₁₉N₅O₃S₂ | 1-Ethyl-N- (4-methyl-3- sulfamoylphenyl)-6- (2-thienyl)-1H- pyrazolo[3,4-b] pyridine- 4-carboxamide | 4.3 | 100% |
| 20 | 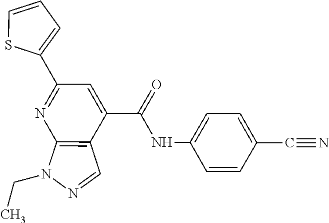 [SMILES: CCn1ncc2c(C(=O)Nc3ccc(C#N)cc3)cc(-c3cccs3)nc21] | C₂₀H₁₅N₅OS | N-(4-Cyanophenyl)- 1-ethyl-6-(2- thienyl)-1H-pyrazolo [3,4-b]pyridine-4- carboxamide | 1.9 | 99.6% |
| 21 | 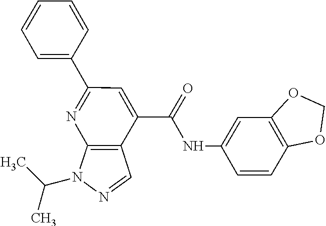 [SMILES: CC(C)n1ncc2c(C(=O)Nc3ccc4c(c3)OCO4)cc(-c3ccccc3)nc21] | C₂₃H₂₀N₄O₃ | N-(1,3-Benzodioxol- 5-yl)-1-isopropyl- 6-phenyl-1H-pyrazolo [3,4-b]pyridine-4- carboxamide | 4.5 | 100% |

Quinoline and derivatives

| | | | | | |
|---|---|---|---|---|---|
| 22 (Mefloquine hydrochloride) | 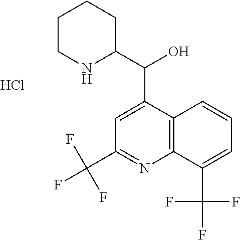 [SMILES: Cl ; OC(c1cc(C(F)(F)F)nc2c(C(F)(F)F)cccc12)C1CCCCN1] | C₁₇H₁₇ClF₆N₂O | [2,8-Bis (trifluoromethyl)- 4-quinolinyl](2- piperidinyl)methanol hydrochloride (1:1) | 4.0 | 100% |
| 23 | 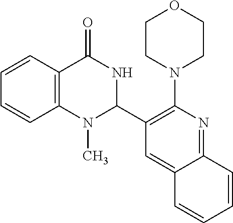 [SMILES: CN1c2ccccc2C(=O)NC1c1cc2ccccc2nc1N1CCOCC1] | C₂₂H₂₂N₄O₂ | 1-Methyl-2-[2-(4- morpholinyl)-3- quinolinyl]-2,3- dihydro-4(1H)- quinazolinone | 4.4 | 100% |
| 24 | 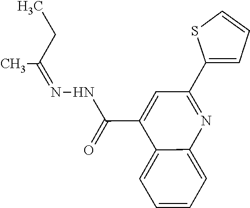 [SMILES: CC/C(C)=N\NC(=O)c1cc(-c2cccs2)nc2ccccc12] | C₁₈H₁₇N₃OS | N′-[(2Z)-2- Butanylidene]- 2-(2-thienyl)- 4- quinoline- carbohydrazide | 4.0 | 100% |
| 25 | 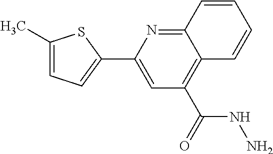 [SMILES: Cc1ccc(-c2cc(C(=O)NN)c3ccccc3n2)s1] | C₁₅H₁₃N₃OS | 2-(5-Methyl-2- thienyl)-4- quinoline- carbohydrazide | 3.7 | 100% |
| 26 | 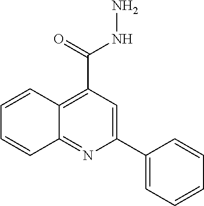 [SMILES: NNC(=O)c1cc(-c2ccccc2)nc2ccccc12] | C₁₆H₁₃N₃O | 2-Phenyl-4- quinoline- carbohydrazide | 2.5 | 98.7% |
| 27 | 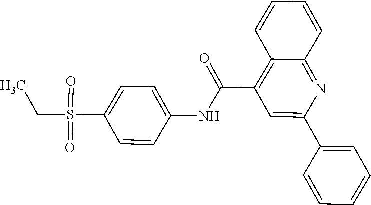 [SMILES: CCS(=O)(=O)c1ccc(NC(=O)c2cc(-c3ccccc3)nc3ccccc23)cc1] | C₂₄H₂₀N₂O₃S | N-[4-(Ethylsulfonyl) phenyl]-2-phenyl-4- quinoline- carboxamide | 3.4 | 89.6% |
| 28 | 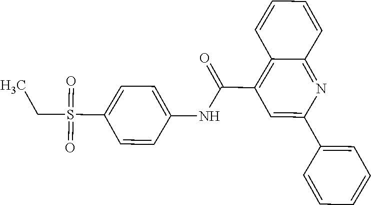 [SMILES: CCS(=O)(=O)c1ccc(NC(=O)c2cc(-c3ccccc3)nc3ccccc23)cc1] | C₂₃H₁₇FN₂O₃S | 2-(4-Fluorophenyl)- N-[4- (methylsulfonyl) phenyl]-4- quinolinecarboxamide | 4.5 | 99.6% |
| 29 | 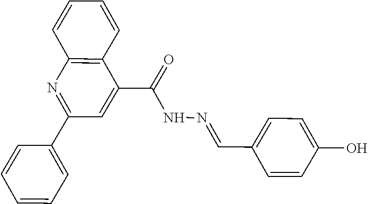 [SMILES: O=C(N/N=C/c1ccc(O)cc1)c1cc(-c2ccccc2)nc2ccccc12] | C₂₃H₁₇N₃O₂ | N′-[(E)-(4- Hydroxyphenyl) methylene]- 2-phenyl-4- quinoline- carbohydrazide | 3.8 | 100% |
| 30 | 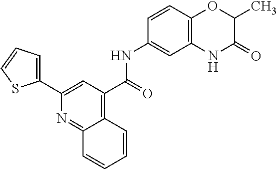 [SMILES: CC1Oc2ccc(NC(=O)c3cc(-c4cccs4)nc4ccccc34)cc2NC1=O] | C₂₃H₁₇N₃O₃S | N-(2-Methyl-3-oxo- 3,4-dihydro-2H-1,4- benzoxazin-6-yl)- 2-(2-thienyl)-4- quinolinecarboxamide | 4.1 | 100% |
| 31 | 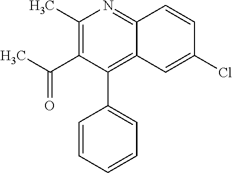 [SMILES: CC(=O)c1c(C)nc2ccc(Cl)cc2c1-c1ccccc1] | C₁₈H₁₄ClNO | 1-(6-Chloro-2- methyl-4-phenyl-3- quinolinyl)ethanone | 3.8 | 94.5% |
| 32 | 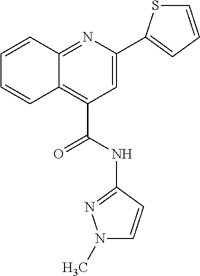 [SMILES: Cn1ccc(NC(=O)c2cc(-c3cccs3)nc3ccccc23)n1] | C₁₈H₁₄N₄OS | N-(1-Methyl-1H- pyrazol-3-yl)-2-(2- thienyl)-4- quinoline- carboxamide | 4.0 | 100% |
| 33 | 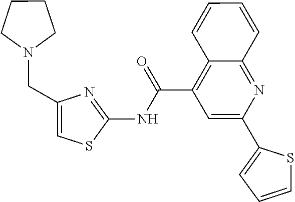 [SMILES: O=C(Nc1nc(CN2CCCC2)cs1)c1cc(-c2cccs2)nc2ccccc12] | C₂₂H₂₀N₄OS₂ | N-[4-(1- Pyrrolidinylmethyl)- 1,3-thiazol- 2-yl)-2-(2-thienyl)-4- quinoline- carboxamide | 4.5 | 100% |
| 34 | 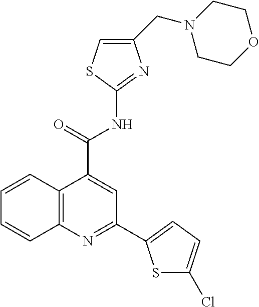 [SMILES: O=C(Nc1nc(CN2CCOCC2)cs1)c1cc(-c2ccc(Cl)s2)nc2ccccc12] | C₂₂H₁₉ClN₄O₂S₂ | 2-(5-Chloro-2- thienyl)-N-[4-(4- morpholinylmethyl)- 1,3-thiazol-2-yl]-4- quinoline- carboxamide | 3.5 | 94.4% |
| 35 (Dibucaine) | 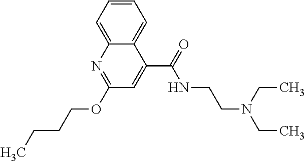 [SMILES: CCCCOc1cc(C(=O)NCCN(CC)CC)c2ccccc2n1] | C₂₀H₂₉N₃O₂ | 2-Butoxy-N-[2- (diethylamino) ethyl]-4- quinoline- carboxamide | N.D. | 100% |
| 36 | 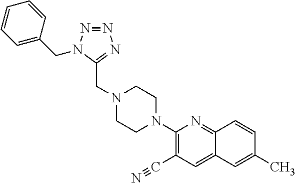 [SMILES: Cc1ccc2nc(N3CCN(Cc4nnnn4Cc4ccccc4)CC3)c(C#N)cc2c1] | C₂₄H₂₄N₈ | 2-{4-[(1-Benzyl- 1H-tetrazol-5- yl)methyl]-1- piperazinyl}-6- methyl-3- quinoline- carbonitnle | 1.0 | 79.7% |
| 37 | 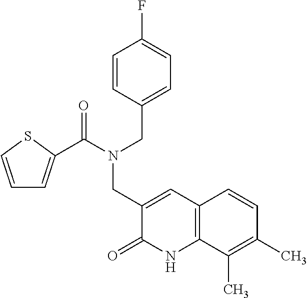 [SMILES: Cc1ccc2cc(CN(Cc3ccc(F)cc3)C(=O)c3cccs3)c(=O)[nH]c2c1C] | C₂₄H₂₁FN₂O₂S | N-[(7,8-Dimethyl-2- oxo-1,2-dihydro-3- quinolinyl)methyl]- N-(4-fluorobenzyl)- 2- thiophene- carboxamide | 4.2 | 99.7% |
| 38 | 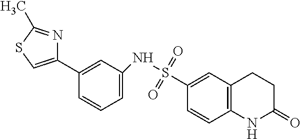 [SMILES: Cc1nc(-c2cccc(NS(=O)(=O)c3ccc4c(c3)CCC(=O)N4)c2)cs1] | C₁₉H₁₇N₃O₃S₂ | N-[3-(2-Methyl-1,3- thiazol-4-yl)phenyl]- 2-oxo-1,2,3,4- tetrahydro-6- quinoline- sulfonamide | 0.9 | 59.1% |

Benzimidazole

| | | | | | |
|---|---|---|---|---|---|
| 39 | 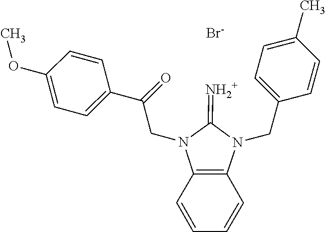 [SMILES: COc1ccc(C(=O)Cn2c(=[NH2+])n(Cc3ccc(C)cc3)c3ccccc32)cc1 ; [Br-]] | C₂₄H₂₄BrN₃O₂ | 1-[2-(4-Methoxyphenyl)- 2-oxoethyl]-3- (4-methylbenzyl)- 1,3-dihydro-2H- benzimidazol-2- iminium bromide | 0.3 | 100% |
| 40 | 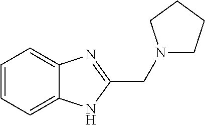 [SMILES: c1ccc2[nH]c(CN3CCCC3)nc2c1] | C₁₂H₁₅N₃ | 2-(1- Pyrrolidinylmethyl)- 1H- benzimidazole | 4.0 | 100% |
| 41 | 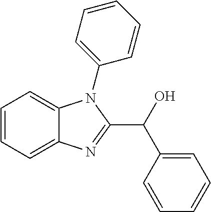 [SMILES: OC(c1ccccc1)c1nc2ccccc2n1-c1ccccc1] | C₂₀H₁₆N₂O | Phenyl(1-phenyl-1H- benzimidazol-2- yl)methanol | 4.1 | 100% |
| 42 | 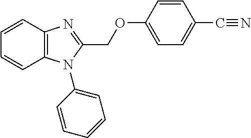 [SMILES: N#Cc1ccc(OCc2nc3ccccc3n2-c2ccccc2)cc1] | C₂₁H₁₅N₃O | 4-[(1-Phenyl- 1H-benzimidazol-2- yl)methoxy) benzonitrile | 4.5 | 100% |
| 43 | 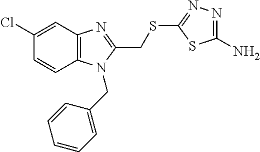 [SMILES: Nc1nnc(SCc2nc3cc(Cl)ccc3n2Cc2ccccc2)s1] | C₁₇H₁₄ClN₅S₂ | 5-{[(1-Benzyl- 5-chloro-1H- benzimidazol-2- yl)methyl]sulfanyl}- 1,3,4-thiadiazol- 2-amine | 0.3 | 91.3% |

1,4-Benzodioxan or 1,2-Ethylenedioxybenzene

| | | | | | |
|---|---|---|---|---|---|
| 44 | 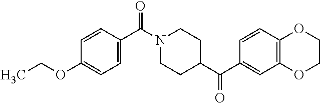 [SMILES: CCOc1ccc(C(=O)N2CCC(C(=O)c3ccc4c(c3)OCCO4)CC2)cc1] | C₂₃H₂₅NO₅ | 2,3-Dihydro-1,4- benzodioxin-6-yl[1-(4- ethoxybenzoyl)-4- piperidinyl] methanone | 0.2 | 84.9% |
| 45 | 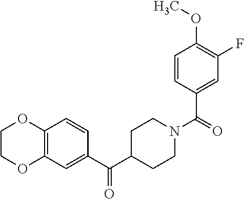 [SMILES: COc1ccc(C(=O)N2CCC(C(=O)c3ccc4c(c3)OCCO4)CC2)cc1F] | C₂₂H₂₂FNO₅ | 2,3-Dihydro-1,4- benzodioxin-6-yl [1-(3-fluoro-4- methoxybenzoyl)-4- piperidinyl] methanone | 2.0 | 82.1% |
| 46 | 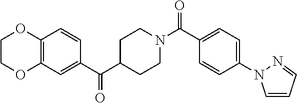 [SMILES: O=C(c1ccc2c(c1)OCCO2)C1CCN(C(=O)c2ccc(-n3cccn3)cc2)CC1] | C₂₄H₂₃N₃O₄ | 2,3-Dihydro-1,4- benzodioxin-6-yl {1-[4-(1H- pyrazol- 1-yl)benzoyl]-4- piperidinyl}methanone | 1.3 | 82.6% |

Quinazolinone

| | | | | | |
|---|---|---|---|---|---|
| 47 | 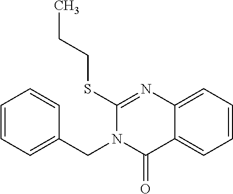 [SMILES: CCCSc1nc2ccccc2c(=O)n1Cc1ccccc1] | C₁₈H₁₈N₂OS | 3-Benzyl-2- (propylsulfanyl)- 4(3H)- quinazolinone | 4.1 | 100% |
| 48 | 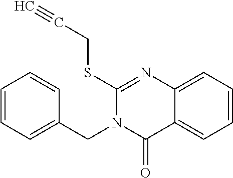 [SMILES: C#CCSc1nc2ccccc2c(=O)n1Cc1ccccc1] | C₁₈H₁₄N₂OS | 3-Benzyl-2-(2- propyn-1-ylsulfanyl)- 4(3H)- quinazolinone | 4.5 | 100% |
| 49 | 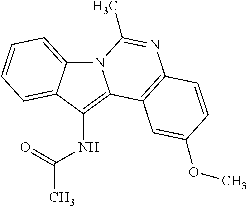 [SMILES: COc1ccc2nc(C)n3c4ccccc4c(NC(C)=O)c3c2c1] | C₁₉H₁₇N₃O₂ | N-(2-Methoxy-6- methylindolo[1,2- c]quinazolin- 12-yl)acetamide | 1.0 | 17.2% |
| 50 | 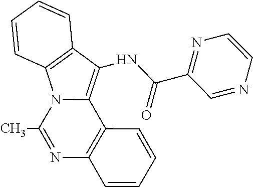 [SMILES: Cc1nc2ccccc2c2c(NC(=O)c3cnccn3)c3ccccc3n12] | C₂₁H₁₅N₅O | N-(6-Methylindolo [1,2-c]quinazolin-12- yl)-2- pyrazinecarboxamide | 2.5 | 69.9% |
| 51 | 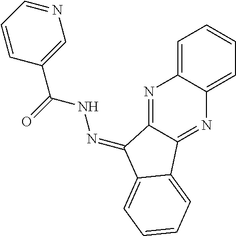 [SMILES: O=C(N/N=C1/c2ccccc2-c2nc3ccccc3nc21)c1cccnc1] | C₂₁H₁₃N₅O | N′-[(11Z)-11H- Indeno[1,2-b] quinoxalin- 11-ylidene] nicotinohydrazide | 0.1 | 1.6% |

Pyrimidine

| | | | | | |
|---|---|---|---|---|---|
| 52 | 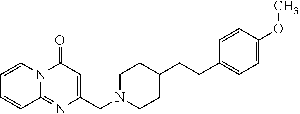 [SMILES: COc1ccc(CCC2CCN(Cc3cc(=O)n4ccccc4n3)CC2)cc1] | C₂₃H₂₇N₃O₂ | 2-({4-[2-(4- Methoxyphenyl) ethyl]-1- piperidinyl}methyl)- 4H-pyrido[1,2- a]pyrimidin-4-one | 0.0 | 57.7% |
| 53 | 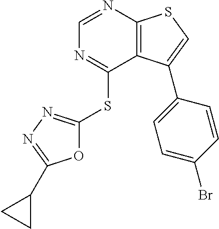 [SMILES: Brc1ccc(-c2csc3ncnc(Sc4nnc(C5CC5)o4)c23)cc1] | C₁₇H₁₁BrN₄OS₂ | 5-(4-Bromophenyl)-4- [(5-cyclopropyl- 1,3,4-oxadiazol-2- yl)sulfanyl]thieno [2,3-d]pyrimidine | 1.8 | 100% |
| 54 | 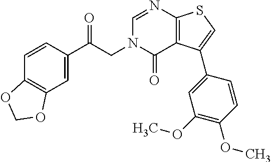 [SMILES: COc1ccc(-c2csc3ncn(CC(=O)c4ccc5c(c4)OCO5)c(=O)c23)cc1OC] | C₂₃H₁₈N₂O₆S | 3-[2-(1,3- Benzodioxol-5-yl)-2- oxoethyl]-5-(3,4- dimethoxyphenyl) thieno[2,3- d]pyrimidin-4 (3H)-one | 3.2 | 87.6% |

Benzoic acid

| | | | | | |
|---|---|---|---|---|---|
| 55 | 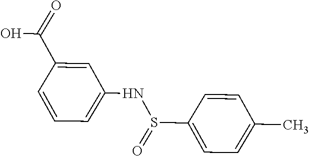 [SMILES: Cc1ccc(S(=O)Nc2cccc(C(=O)O)c2)cc1] | C₁₄H₁₃NO₃S | 3-{[(4-Methylphenyl) sulfinyl]amino} benzoic acid | 2.5 | 98.4% |
| 56 | 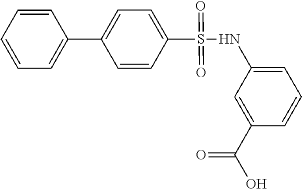 [SMILES: O=C(O)c1cccc(NS(=O)(=O)c2ccc(-c3ccccc3)cc2)c1] | C₁₉H₁₅NO₄S | 3-[(4- Biphenylylsulfonyl) amino] benzoic acid | 0.3 | 85.4% |

Furamide

| | | | | | |
|---|---|---|---|---|---|
| 57 | 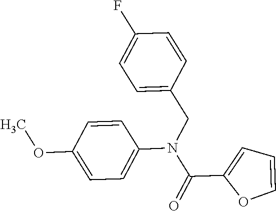 [SMILES: COc1ccc(N(Cc2ccc(F)cc2)C(=O)c2ccco2)cc1] | C₁₉H₁₆FNO₃ | N-(4-Fluorobenzyl)- N-(4- methoxyphenyl)- 2-furamide | 4.3 | 99.7% |
| 58 | 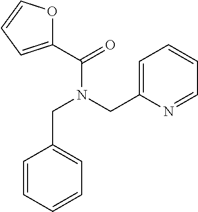 [SMILES: O=C(c1ccco1)N(Cc1ccccc1)Cc1ccccn1] | C₁₈H₁₆N₂O₂ | N-Benzyl-N- (2-pyridinylmethyl)-2- furamide | 2.4 | 91.7% |

Imidazo[1,2-a]pyrazine

| | | | | | |
|---|---|---|---|---|---|
| 59 | 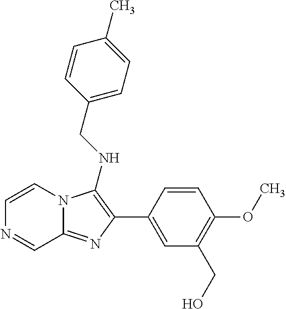 [SMILES: COc1ccc(-c2nc3cnccn3c2NCc2ccc(C)cc2)cc1CO] | C₂₂H₂₂N₄O₂ | (2-Methoxy- 5-{3-[(4- methylbenzyl) amino]imidazo[1,2- a]pyrazin-2-yl} phenyl)methanol | 1.3 | 75.2% |
| 60 | 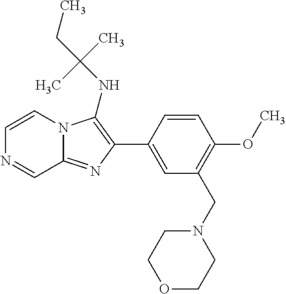 [SMILES: CCC(C)(C)Nc1c(-c2ccc(OC)c(CN3CCOCC3)c2)nc2cnccn12] | C₂₃H₃₁N₅O₂ | 2-[4-Methoxy-3-(4- morpholinylmethyl) phenyl]-N-(2- methyl-2-butanyl) imidazo[1,2- a]pyrazin-3-amine | 1.8 | 100% |

Other antiviral agents

| | | | | | |
|---|---|---|---|---|---|
| 61 | 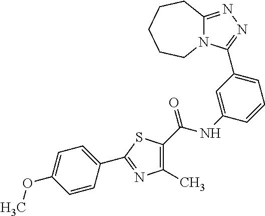 [SMILES: COc1ccc(-c2nc(C)c(C(=O)Nc3cccc(-c4nnc5n4CCCCC5)c3)s2)cc1] | C₂₅H₂₅N₅O₂S | 2-(4-Methoxyphenyl)- 4-methyl-N-[3- (6,7,8,9-tetrahydro- 5H- [1,2,4]triazolo [4,3-a]azepin-3- yl)phenyl]-1,3- thiazole-5- carboxamide | 1.8 | 96.0% |
| 62 | 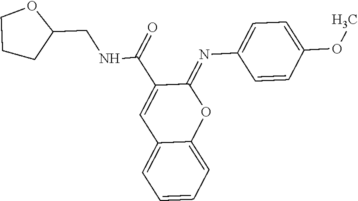 [SMILES: COc1ccc(/N=c2\oc3ccccc3cc2C(=O)NCC2CCCO2)cc1] | C₂₂H₂₂N₂O₄ | (2Z)-2-[(4- Methoxyphenyl)imino]- N- (tetrahydro-2- furanylmethyl)-2H- chromene-3- carboxamide | 1.5 | 90.4% |
| 63 | 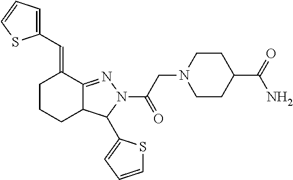 [SMILES: NC(=O)C1CCN(CC(=O)N2N=C3/C(=C/c4cccs4)CCCC3C2c2cccs2)CC1] | C₂₄H₂₈N₄O₂S₂ | 1-{2-Oxo-2-[(7E)-3- (2-thienyl)-7-(2- thienylmethylene)- 3,3a,4,5,6,7- hexahydro-2H- indazol-2-yl]ethyl}-4- piperidine- carboxamide | 4.2 | 100% |
| 64 | 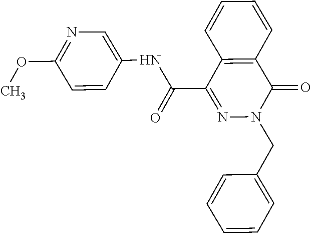 [SMILES: COc1ccc(NC(=O)c2nn(Cc3ccccc3)c(=O)c3ccccc23)cn1] | C₂₂H₁₈N₄O₃ | 3-Benzyl-N-(6-methoxy- 3-pyridinyl)-4- oxo-3,4-dihydro-1- phthalazine- carboxamide | 4.2 | 100% |
| 65 | 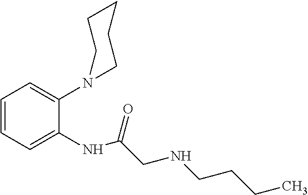 [SMILES: CCCCNCC(=O)Nc1ccccc1N1CCCCC1] | C₁₇H₂₇N₃O | N²-Butyl-N-[2-(1- piperidinyl)phenyl] glycinamide | −0.4 | 36.2% |
| 66 | 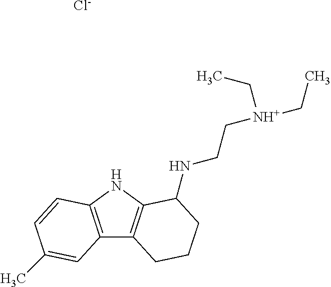 [SMILES: [Cl-] ; CC[NH+](CC)CCNC1CCCc2c1[nH]c1ccc(C)cc21] | C₁₉H₃₀ClN₃ | N,N-Diethyl-2- [(6-methyl-2,3,4,9- tetrahydro- 1H-carbazol-1- yl)amino] ethanaminium chloride | 4.4 | 99.6% |
| 67 | 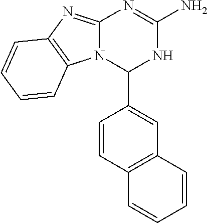 [SMILES: NC1=Nc2nc3ccccc3n2C(c2ccc3ccccc3c2)N1] | C₁₉H₁₅N₅ | 4-(2-Naphthyl)-1,4- dihydro[1,3,5] triazino[1,2- a]benzimidazol- 2-amine | 1.9 | 67.7% |
| 68 | 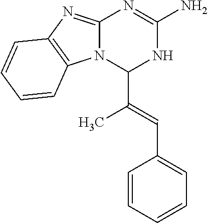 [SMILES: C/C(=C\c1ccccc1)C1NC(N)=Nc2nc3ccccc3n21] | C₁₈H₁₇N₅ | 4-[(1E)-1-Phenyl-1- propen-2-yl)-1,4- dihydro[1,3,5] triazino[1,2- a]benzimidazol- 2-amine | 3.7 | 100% |
| 69 | 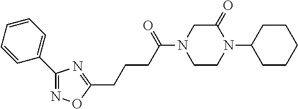 [SMILES: O=C(CCCc1nc(-c2ccccc2)no1)N1CCN(C2CCCCC2)C(=O)C1] | C₂₂H₂₈N₄O₃ | 1-Cyclohexyl-4- [4-(3-phenyl-1,2,4- oxadiazol-5- yl)butanoyl]-2- piperazinone | 2.0 | 96.5% |
| 70 (Flupentixol dihydrochloride cis-(Z)) | 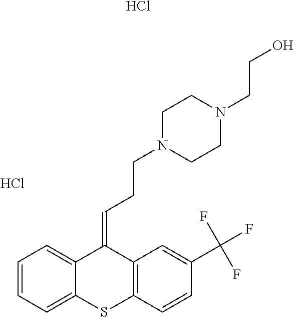 [SMILES: Cl ; Cl ; OCCN1CCN(CC/C=C2/c3ccccc3Sc3ccc(C(F)(F)F)cc32)CC1] | C₂₃H₂₇Cl₂F₃N₂OS | 2-(4-{(3Z)-3-[2- (Trifluoromethyl)-9H- thioxanthen-9- ylidene]propyl}-1- piperazinyl)ethanol dihydrochloride | 3.9 | 99.6% |
| 71 (Zuclopenthixol hydrochloride) | 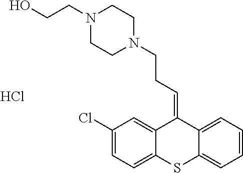 [SMILES: Cl ; OCCN1CCN(CC/C=C2/c3ccccc3Sc3ccc(Cl)cc32)CC1] | C₂₂H₂₆Cl₂N₂OS | 2-[4-[3-(2- chlorothioxanthen-9- ylidene)propyl] piperazin- 1-yl]ethanol hydrochloride | 4.2 | 99.1% |
| 72 (Fluoxetine hydrochloride) | 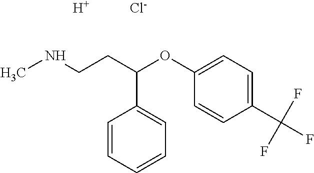 | C₁₇H₁₉ClF₃NO | N-Methyl-3- phenyl-3-[4- (trifluoromethyl) phenoxy]-1- propanamine hydrochloride (1:1) | 4.7 | 99.7% |
| 73 (Lycorine hydrochloride) | 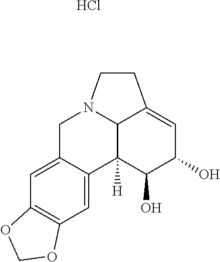 [SMILES: Cl ; O[C@@H]1[C@@H](O)C=C2CCN3Cc4cc5c(cc4[C@H]1C23)OCO5] | C₁₆H₁₈ClNO₄ | (1S,2S,12bS)- 2,4,5,7,12b,12c- Hexahydro-1H- [1,3]dioxolo[4,5- j]pyrrolo[3,2,1-de] phenanthridine-1,2- diol hydrochloride (1:1) | 2.8 | 100% |
| 74 (Norfluoxetine) | 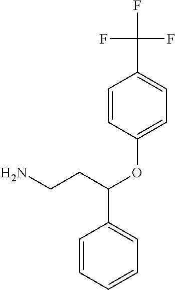 [SMILES: NCCC(Oc1ccc(C(F)(F)F)cc1)c1ccccc1] | C₁₆H₁₆F₃NO | 3-Phenyl-3-[4- (trifluoromethyl) phenoxy]-1- propanamine | 4.7 | 100% |

### Antiviral Activity Quantification:

a) A compound was added to HeLa-RW cells 30 minutes before they were infected with CVB3-H3 at multiplicity of infection of 1. To control wells only DMSO was added. After 6 hours, cells were washed twice, trypsinized, lysed by 3 freeze thaw cycles, and clarified. Serial-ten fold dilutions were prepared and applied to cells for standard 2 day plaque reduction assays. Plaques were manually counted. Titer values are expressed as plaque forming units per ml. “Log₁₀ reduction”=log₁₀ (Viral titer with DMSO only/Viral titer with inhibitor).
b) HeLa-RW cells were plated at 30,000 cells per 100 μl per well in a 96-well microtiter plate and cultured overnight. The next day, the cells were infected with eGFP-expressing recombinant CVB3 at m.o.i=1. A compound was added to the cells at the time of infection. At the 6 hr post infection, the cells were fixed with 1% PFA. For counterstain, the fixed cells were stained with DAPI for 5 minutes. The cells were washed 3 times with PBS before being analyzed using an ImageXpress Micro high content microscope (Molecular Devices). Replication of eGFP-CVB3 was determined by the percentage of eGFP⁺ cells/DAPI⁺ cells. Inhibition of eGFP-CVB3 infecting cells was defined by the formula: 1−(% of eGFP⁺ cells with a compound/% of eGFP⁺ cells with DMSO only).

Norfluoxetine is a known human metabolite of fluoxetine and is present in near equimolar concentrations with fluoxetine during therapy with recommended doses for depression and other conditions.

### Example 1

### Examination of Effective Concentrations and Cytotoxicity

Procedure: Enteroviruses rapidly cause cell death, and a cell viability assay was used to examine the cytotoxicity of each compound and to identify the concentration needed to prevent cell death caused by coxsackievirus B3 (CVB3) infection.

Compounds were diluted two-fold and added to wells of 384 well plate. HeLa-RW cells were then added (cytotoxicity testing), or cells and CVB3 were added to provide a low multiplicity of infection to determine concentration of peak activity or 50% maximal concentration. Selectivity index is defined as the 50% Cytotoxic concentration/50% activity concentration

TABLE 2
Determination of effective concentrations using cell viability assay

| Com- | 50% | Peak | 50% activity | Selec- |
|---|---|---|---|---|
| pound | Cytotoxic | Activity | concentration | tivity |
| ID | concentration | Conc. (μM) | (IC50) (μM) | index |
| 57 | 50 | 6.25 | 3.13 | 16 |
| 25 | 25 | 3.13 | 0.78 | 32 |
| 1 | 25 | 1.56 | 0.60 | 42 |
| 23 | 50 | 3.13 | 0.92 | 54 |
| 21 | 25 | 3.13 | 1.19 | 21 |
| 16 | 25 | 3.13 | 1.15 | 22 |
| 68 | 25 | 3.13 | 1.21 | 21 |
| 45 | 25 | 1.56 | 0.20 | 125 |
| 42 | 25 | 6.25 | 3.0 | 8 |
| 3 | 200 | 3.13 | 1.14 | 175 |
| 33 | 25 | 3.13 | 0.57 | 44 |
| Fluoxetine | 25 | 6.25 | 2.34 | 11 |
| Mefloquine | 10 | 3.13 | 1.60 | 6 |
| Norfluoxetine | 25 | 6.25 | 2.53 | 10 |

Note that the IC₅₀ values estimated for fluoxetine (2.34 mM) and its metabolite norfluoxetine (2.53 μM) are highly similar. Concentrations of 1000 μM (1 mM) are achieved during chronic therapy 80 mg/day fluoxetine (see, for e.g., Henry M E, et al., A comparison of brain and serum pharmacokinetics of R-fluoxetine and racemic fluoxetine: A 19-F MRS study. Neuropsychopharmacology. 2005 Aug.; 30(8): 1576-83) are comparable to the average steady state concentrations during chronic therapy for depression.

### Example 2

### Quantitation of Antiviral Activity by Plaque Reduction Assay

Procedure:

Compounds were added HeLa-RW cells 30 minutes before they were infected with CVB3-H3 at multiplicity of infection of 1. Control wells were not treated with compound. After 6 hours, cells were washed twice, trypsinized, lysed by 3 freeze thaw cycles, and clarified. Serial-ten fold dilutions were prepared and applied to cells for standard 2 day plaque reduction assays. Plaques were manually counted. Titer values are expressed as plaque forming units per ml. “Log₁₀ reduction”=log₁₀ (Viral titer without inhibitor/Viral titer with inhibitor). Results are shown in Table 1. Column: Antiviral Activity/Log₁₀ reduction of virus titer.

Most compounds reduced viral titer by more than 4 log₁₀ units (more than 10,000 fold, see Table 1).

### Example 3

### Determination of Antiviral Activity by RT-PCR Assay

Procedure:

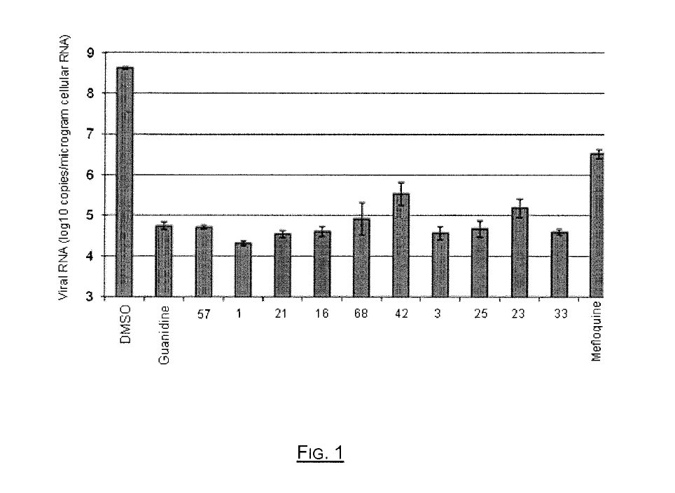

To assess the effect on viral RNA accumulation in cells, HeLa RW cells were infected with CVB-H3 after 30 minutes pretreatment with each compound at the concentrations indicated in Table 1. After six hours, the cells were washed twice, harvested by trypsinization, washed again, and viral RNA was quantified by real-time reverse transcriptase PCR (RT-PCR) using synthetic RNA as a template for control standards. The solvent DMSO served as a negative control (no antiviral activity) while guanidine, a potent inhibitor of CVB3 replication, served as a positive control. Experiments were performed with triplicate wells in two separate infections. These results are depicted in FIG. 1.

These data indicate that amount of viral RNA in infected cells was reduced by 3 orders of magnitude or more by most compounds.

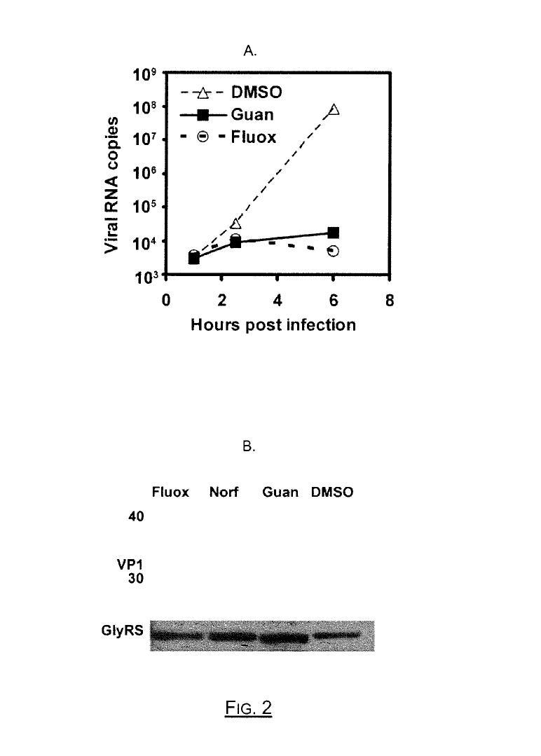

This result for fluoxetine is corroborated by a similar experiment (FIG. 2 A) with additional time points at 1,2.5, and 6 hours. The similarity in the amount of viral RNA detected at 1 hour after inoculation of cells at 2.5 hours for fluoxetine and guanidine treated cells indicates that the entry of CVB3 virions was not impeded by fluoxetine. These data suggest instead that fluoxetine has a mechanism of antiviral activity involving some intracellular step in enterovirus replication. Furthermore, Immunoblot revealed a total abrogation of detectable viral protein synthesis (panel B).

### Example 4

### Determination of Antiviral Activity by Inhibition of eGFP-Expressing Recombinant CVB3

Procedure:

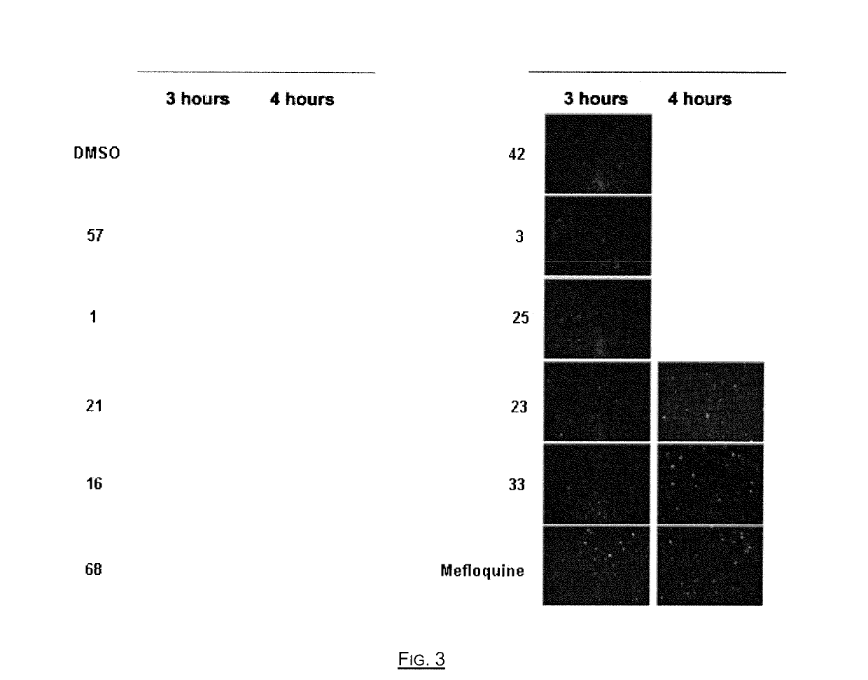

HeLa-RW cells were plated at 30,000 cells per 100 μl per well in a 96-well microtiter plate and cultured overnight. The next day, the cells were infected with eGFP-expressing recombinant CVB3 at m.o.i=1. The results of inhibition of infection of eGFP-CVB3 are shown in Table 1: Column Antiviral Activity/inhibition of infected cells. In addition, 14 compounds were added to the cells at 0 hr, 1 hr and 2 hr post infection at concentrations of their peak activities indicated by the IC₅₀ experiment. At the 6 hr post infection, the cells were fixed with 1% PFA. For counterstain, the fixed cells were stained with DAPI for 5 minutes. The cells were washed 3 times with PBS before being analyzed using an ImageXpress Micro high content microscope (Molecular Devices). See FIG. 3 for photomicrographs. The figure demonstrates that CVB3 replication is inhibited by the compounds indicated. DMSO serves as a negative control.

TABLE 3
Inhibition of eGFP-expressing recombinant CVB3

| | |
|---|---|
| Concentration | % of eGFP + cells |

| Compound | used (μM) | 0 Hr | 1 Hr | 2 Hr |
|---|---|---|---|---|
| 57 | 6.25 | 0.0 | 0.0 | 0.0 |
| 25 | 3.13 | 0.1 | 0.1 | 0.0 |
| 1 | 1.56 | 0.0 | 0.0 | 0.0 |
| 23 | 3.13 | 0.1 | 3.8 | 0.0 |
| 21 | 3.13 | 0.0 | 0.0 | 0.0 |
| 16 | 3.13 | 0.0 | 0.1 | 0.0 |
| 68 | 3.13 | 0.0 | 0.0 | 0.0 |
| 45 | 1.56 | 8.9 | 17.0 | 20.0 |
| 42 | 6.25 | 0.0 | 0.1 | 0.0 |
| 3 | 3.13 | 0.0 | 0.0 | 0.0 |
| Fluoxetine | 6.25 | 0.0 | 0.0 | 0.0 |
| 33 | 3.13 | 0.0 | 0.0 | 0.0 |
| Mefloquine | 3.13 | 21.1 | 18.5 | 27.9 |
| Norfluoxetine | 6.25 | 0.0 | 0.0 | 0.0 |
| DMSO | | 26.0 | 23.7 | 24.8 |

### Example 5

### Test of Antiviral Activity Against Other Enterovirus Strains

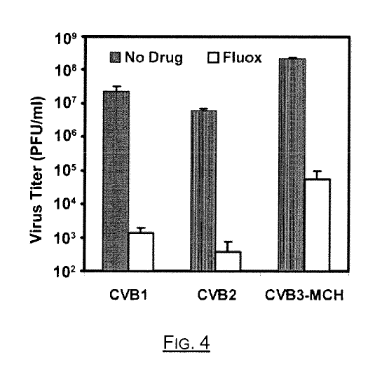

Applicants have examined the antiviral activity of fluoxetine against two other coxsackievirus strains using an experimental design described in section III. Wells of HeLa-RW were infected with patient derived isolates of coxsackievirus B1, (CVB1) coxsackievirus B2 (CVB2) or coxsackievirus B3-MCH (CVB3-MCH) (See for e.g. Krogstad, Hammon, Halnon, and Whitton. Pediatr Infect Dis J. 2008 Jul.; 27(7):668-9) with or without exposure to fluoxetine. As seen in FIG. 4, fluoxetine reduced the replication of these additional enterovirus strains by 4.2, 4.2, and 3.6 log 10 units (10,000 and more than 4,000 fold respectively).

### Example 6

### Immunoblot Demonstration of Antiviral Activity of Selected Compounds

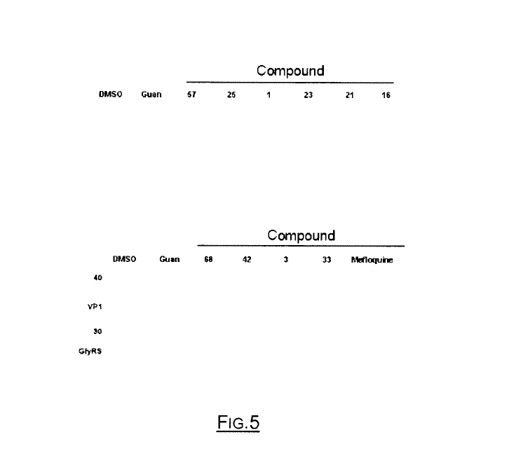

The experiment shown in FIG. 5 below demonstrates the antiviral activity of the designated compounds by showing a reduction in the amount of viral protein made detected 6 hours after inoculation of HeLa cell cells with CVB3. Compounds were added 30 minutes before inoculation of cell cultures with virus. Immunoblot with antibody directed against the viral capsid proteins of CVB3 was used to demonstrate the loss of detectable viral protein in cells treated with compounds designated. The solvent DMSO served as a negative control (no antiviral activity) while the compound guanidine, a potent inhibitor of CVB3 replication, served as a positive control.

### Example 7

### Inhibition of Polioviruses by Compound 3

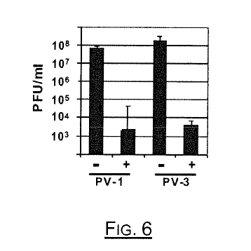

Among the pyrazolo [3,4-b]pyridine compounds examined, compound 3 also inhibited the replication of poliovirus-1 and -3 (FIG. 6), which are genetically distinct enteroviruses from the coxsackievirus B 3 used for screening. HeLa cells were infected at an MOI of 1 with poliovirus 1 (PV-1) or poliovirus-3 (PV-3) with or without 10 mM compound 3 in the medium. After six hours the cells were lysed and titer of virus produced was determined by standard plaque assay. A marked reduction of titer with exposure (+) to compound 3 was observed.

### Example 8

### Time of Addition Experiment

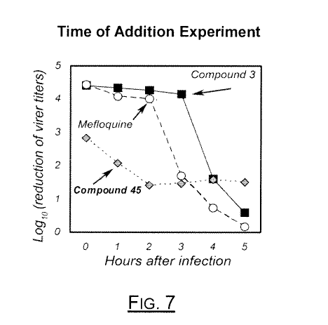

The compounds utilized herein vary in antiviral activity when examined in plaque reduction assays: addition of compounds 45 and mefloquine reduced the amount of virus produced in single cycle growth experiments by 1.3 to 2.8 log₁₀ plaque form units (PFU)/ml (20 to 600 fold). Compound 45 inhibited CVB3 replication in HeLa cells with a 50% effective concentration (EC50) of 200 nM, and was minimally cytotoxic to HeLa cells. Compound 45 markedly reduced CVB3 replication when added prior to inoculation of cultures (FIG. 7), but was less potent if added 1 hour or more after inoculation. These data show that these compounds inhibit CVB3 replication by either destabilizing virion structure or by blocking viral entry. These data show distinct mechanisms of action for compound 45 compared to compound 3, which points to synergistic antiviral activity if these were used in combination.

Applicants have reproducibly demonstrated antiviral activity of 74 compounds identified by high-throughput screening using plaque reduction and RT-PCR assays with CVB3. In addition, their antiviral activity was demonstrated by quantifying their inhibition of the expression of EGFP (enhanced green fluorescent protein) expression in cells infected via a recombinant CVB3 variant containing the EGFP gene. Applicants have also shown that fluoxetine inhibits three additional enteroviruses, CVB1, CVB2 and CVB3-MCH. Moreover, these data demonstrate that norfluoxetine, a metabolite of fluoxetine, also exhibits antiviral activity.

Throughout this disclosure, various publications, patents and/or published patent specifications are referenced by an identifying citation. The disclosures of these publications, patents and published patent specifications are hereby incorporated by reference into the present disclosure to more fully describe the state of the art to which this invention pertains.

1. A method for inhibiting the replication of enterovirus in a cell infected with enterovirus, comprising contacting an effective amount of a compound selected from the group consisting of a compound of Formula I, Formula II, Formula IIIa, Formula IIIb, Formula IV, with the cell infected with said virus, or a compound of Table 1, thereby inhibiting the replication of enterovirus in a cell infected with enterovirus.

2. A method for inhibiting death of a cell infected with an enterovirus, comprising contacting the cell with an effective amount of a compound selected from the group consisting of a compound of Formula I, Formula II, Formula IIIa, Formula IIIb, Formula IV, or a compound of Table 1, thereby inhibiting cell death of said cell infected with enterovirus.

3. A method for treating a patient or animal infected with an enterovirus infection comprising administering to said patient or animal an effective amount of a compound selected from the group consisting of a compound of Formula I, Formula II, Formula IIIa, Formula IIIb, Formula IV, or a compound of Table 1, thereby treating the enteroviral infection.

4. The method of claim 1, wherein the compound is of Formula I or is selected from compounds 1-21 of Table 1.

5. The method of claim 2, wherein the compound is of Formula I or is selected from compounds 1-21 of Table 1.

6. The method of claim 3, wherein the compound is of Formula I or is selected from compounds 1-21 of Table 1.

7. The method of claim 4, further comprising administering an effective amount of a compound of Formula IV or a compound selected from compounds 44-46 of Table 1.

8. The method of claim 4, further comprising administering an effective amount of a compound of Formula IV or a compound selected from compounds 44-46 of Table 1.

9. The method of claim 4, further comprising administering an effective amount of a compound of Formula IV or a compound selected from compounds 44-46 of Table 1.

10. The method of claim 1, wherein the compound is of Formula IV or is selected from compounds 44-46 of Table 1.

11. The method of claim 2, wherein the compound is of Formula IV or is selected from compounds 44-46 of Table 1.

12. The method of claim 3, wherein the compound is of Formula IV or is selected from compounds 44-46 of Table 1.

13. The method of claim 10, further comprising administering an effective amount of a compound of Formula I or a compound selected from compounds 1-21 of Table 1.

14. The method of claim 11, further comprising administering an effective amount of a compound of Formula I or a compound selected from compounds 1-21 of Table 1.

15. The method of claim 12, further comprising administering an effective amount of a compound of Formula I or a compound selected from compounds 1-21 of Table 1.

16. A unit dosage form for one or more of:
a) inhibiting the replication of enterovirus in a cell infected with enterovirus;
b) inhibiting cell death of a cell infected with enterovirus;
c) treating an enteroviral infection;
the unit dosage form comprising an effective amount of one or more compounds selected from the group consisting of a compound of Formula I, Formula II, Formula IIIa, Formula IIIb, Formula IV, or a compound of Table 1 and a pharmaceutically acceptable carrier or excipient.

17. A composition comprising one or more compounds selected from the group consisting of a compound of Formula I, Formula II, Formula IIIa, Formula IIIb, Formula IV, or a compound of Table 1 and a pharmaceutically acceptable carrier.
